(19) United States
(12) Patent Application Publication
(10) Pub. No.: US 2026/0028571 A1
(43) Pub. Date: Jan. 29, 2026

(54) BIOREACTORS FOR PERFUSING CELLS

(71) Applicant: Ramot at Tel-Aviv University Ltd., Tel-Aviv (IL)

(72) Inventors: Ronit SATCHI-FAINARO, Tel-Aviv (IL); Lena NEUFELD, Tel-Aviv (IL)

(73) Assignee: Ramot at Tel-Aviv University Ltd., Tel-Aviv (IL)

(21) Appl. No.: 19/349,013

(22) Filed: Oct. 3, 2025

Related U.S. Application Data
(63) Continuation of application No. PCT/IL2024/050350, filed on Apr. 4, 2024.

(30) Foreign Application Priority Data
Apr. 4, 2023 (IL) 301958

(51) Int. Cl. C12M 1/00; C12M 1/12; C12M 3/00; G01N 33/50

(52) U.S. Cl. CPC C12M 29/10; C12M 21/08; C12M 23/40; C12M 23/58; C12M 25/14; G01N 33/5088

(57) ABSTRACT

Cell perfusion systems are disclosed which comprise: a sequence of at least two cell-culture wells comprising: a first cell-culture well which comprises a first population of cells; a second cell-culture well which comprises a second. non-identical population of cells; and a fluid pathway interconnecting the cell culture wells.

The work leading to this has received funding from the following European Union ERC Programs: 2014-2019 under grant agreement No. 617445; 2019-2025 under grant agreement No. 835227 and 2019-2021 under grant agreement No. 862580.

### RELATED APPLICATIONS

This application is a Continuation of PCT Patent Application No. PCT/IL2024/050350 having International filing date of Apr. 4, 2024, which claims the benefit of priority of Israeli Patent Application No. 301958 filed on Apr. 4, 2023. The contents of the above applications are all incorporated by reference as if fully set forth herein in their entirety.

### FIELD AND BACKGROUND OF THE INVENTION

The present invention, in some embodiments thereof, relates to cell perfusion systems having a multi-well platform for use in carrying out perfusion studies.

Much of our understanding of cell biology and the molecular mechanisms that control cell growth, differentiation, and function in health and disease is a result of studying cells cultured on flat polystyrene substrates in flasks and dishes. However, within such environments, tissue architecture is lost, cell-cell interactions are reduced, and cells adapt abnormally to their two-dimensional (2D) surroundings by flattening and altering their gene transcription, protein translation, and functional phenotype. In contrast, three-dimensional (3D) cultures more closely resemble cells in real tissues and, as a consequence, show significantly enhanced structure and function. To further enhance the cell-culture environment, it is important to consider other factors such as the maintenance of the culture conditions over time. Tissues and organs of the body are continuously perfused by the blood circulatory and lymphatic systems, which together ensure a constant refreshment of nutrients and oxygen as well as removal of waste products. In conventional cell-culture, there is no equivalent arrangement, other than the frequent manual changing of the culture media. Whilst the circulatory system in real tissues is dynamic, cell-culture media do not circulate or perfuse about the cells, resulting in a static model primarily reliant upon the diffusion of molecules. Furthermore, nutrients, gases and waste product levels are artificially regulated as a consequence of media changing. This can result in uneven homeostasis and metabolic shock to cells exposed to changes in used and fresh culture media. Perfusion of culture media can overcome some of these issues and further augment the culture model. Such dynamic flow can also be controlled to reduce unstirred layers to improve diffusion and introduce shear stresses to affect cell behavior.

Background art includes Neufeld et al., Sci. Adv. 2021; August 18;7(34):eabi9119. doi: 10.1126/sciadv(dot)abi9119; Gu et al., PNAS, 2004, Volume 101. No. 45, pages 15861-15866; WO 2006/033935 Absbio, Perfusion protocol and (www(dot)resources(dot)amsbio(dot)com/Datasheets/Perfusion-Protocol(dot)pdf).

### SUMMARY OF THE INVENTION

According to an aspect of the invention there is provided a cell-perfusion system comprising:
a sequence of at least two cell-culture wells comprising:
a first cell-culture well which comprises a first population of cells;
a second cell-culture well which comprises a second, non-identical population of cells; and
a fluid pathway interconnecting the first cell-culture well and the second cell-culture well so as to allow for fluid to flow through the sequence in a sequential manner.

According to embodiments of the invention, the fluid pathway comprises at least one channel.

According to embodiments of the invention, the channel is lined with cellular matter.

According to embodiments of the invention, the cellular matter comprises cells selected from the group consisting of endothelial cells, pericytes and smooth muscles cells.

According to embodiments of the invention, at least one of the first and the second population of cells form a 3-dimensional (3D) cellular structure in the respective wells.

According to embodiments of the invention, the 3D cellular structure is a tumor model comprising a plurality of cell types.

According to embodiments of the invention, at least a portion of the plurality of cell types are brain cells.

According to embodiments of the invention, the brain cells are selected from the group consisting of astrocytes, microglia cells, neuronal cells, and glioblastoma cells.

According to embodiments of the invention, at least one of the first and the second population of cells form a 3-dimensional (3D) cellular healthy organ model in the respective wells.

According to embodiments of the invention, at least a part of the cellular structure is generated by 3D printing.

According to embodiments of the invention, the cells of the cellular structure are embedded in an extracellular matrix.

According to embodiments of the invention, the extracellular matrix comprises a synthetic material.

According to embodiments of the invention, the extracellular matrix is naturally occurring.

According to embodiments of the invention, the first population of cells comprises cancer cells and the second population of cells comprises lymph node cells.

According to embodiments of the invention, the first population of cells comprise cancer cells of a tissue of a first subject and the second population of cells comprise cancer cells of an identical tissue of a second subject.

According to embodiments of the invention, the first population of cells comprises cancer cells of a tumor and the second population of cells comprises metastasized cells of the tumor.

According to embodiments of the invention, the first population of cells comprise lymph node cells and the second population of cells comprise metastasized cells of a tumor.

According to embodiments of the invention, the metastasized cells are selected from the group consisting of liver cells, lung cells, bone cells, and brain cells.

According to embodiments of the invention, the system further comprises the fluid, wherein the fluid comprises blood cells.

According to embodiments of the invention, the fluid is selected from the group consisting of blood, serum, plasma and buffy coat.

According to embodiments of the invention, the blood cells comprise peripheral blood mononuclear cells (PBMCs) or buffy coat.

According to embodiments of the invention, the first population or the second population of cells are derived from a subject and the fluid is derived from the subject.

According to embodiments of the invention, the sequence comprises at least three cell-culture wells, wherein a third culture well of the three cell-culture well comprises a third population of cells, wherein the second cell-culture well is connected to the third cell-culture well so as to allow for fluid to flow through the sequence in a sequential manner.

According to embodiments of the invention, the first population of cells comprise cancer cells of a tumor, the second population of cells comprise lymph node cells and the third population of cells comprise metastasized cells of the tumor.

According to embodiments of the invention, the system further comprises an additional sequence of at least two cell-culture wells, wherein a cell-culture well of the first sequence is not in fluid connection with a cell-culture well of the second sequence.

According to embodiments of the invention, the system comprises a microfluidic device.

According to embodiments of the invention, the microfluidic device is connected to a source of the fluid via a peristaltic pump or a micro-pump.

According to embodiments of the invention, the microfluidic device further comprises at least one sensor for monitoring at least one parameter of the fluid.

According to an aspect of the invention there is provided a cell-perfusion system comprising:
a sequence of at least two cell-culture wells comprising:
a first cell-culture well which comprises a first population of cells derived from a test subject;
a second cell-culture well which comprises a second population of cells derived from the test subject;
a fluid pathway interconnecting the first cell-culture well and the second cell-culture well so as to allow for fluid to flow through the sequence in a sequential manner; and
fluid of the fluid pathway, wherein the fluid is derived from the test subject.

According to embodiments of the invention, the first population of cells is identical to the second population of cells.

According to embodiments of the invention, the first population of cells is non-identical to the second population of cells.

According to embodiments of the invention, the first population of cells and/or the second population of cells are derived from a tumor of the subject.

According to embodiments of the invention, at least one of the first of the second population of cells form a 3-D structure in the respective wells.

According to embodiments of the invention, the fluid comprises blood cells.

According to embodiments of the invention, the fluid is selected from the group consisting of serum, plasma, blood and buffy coat.

According to embodiments of the invention, the blood cells comprise blood mononuclear cells (PBMCs).

According to embodiments of the invention, the sequence comprises at least three cell-culture wells.

According to embodiments of the invention, the cell perfusion system comprises an additional sequence of at least two cell-culture wells, wherein a cell-culture well of the first sequence is not in fluid connection with a cell-culture well of the second sequence.

According to an aspect of the invention there is provided a method of determining an effect of an agent comprising:
(a) flowing the agent through the cell perfusion system described herein; and
(b) measuring:
   (i) at least one parameter of the fluid; and/or
   (ii) at least one parameter of the cells of the first and/or second population;
      wherein a change in the at least one parameter of the fluid and/or of the cells as compared to the at least one parameter in the absence of the agent, is indicative of an effect of the agent.

According to embodiments of the invention, the effect is a therapeutic effect.

According to embodiments of the invention, the effect is a prophylactic effect.

According to embodiments of the invention, the effect is a toxic effect.

According to embodiments of the invention, the parameter of the fluid is selected from the group consisting of oxygen concentration, glucose concentration, fatty acids composition, electrical resistance, pH, temperature, metabolite concentration and flow rate.

According to yet another aspect of the invention there is provided a cellularized 3D structure, generated by 3D printing, comprising:
at least one inner compartment comprising B cells;
an outer compartment comprising a first cell type of a lymph node; and
an intermediate compartment, between the inner compartment and the outer compartment comprising a second cell type of the lymph node,
at least one inlet port and at least one exit port.

According to embodiments of the invention, the outer compartment is essentially devoid of B cells.

According to embodiments of the invention, the first cell type comprise macrophages or dendritic cells.

According to embodiments of the invention, the second cell type comprise T cells or dendritic cells.

According to embodiments of the invention, the structure is surrounded by a frame fabricated from a biocompatible elastomer.

According to embodiments of the invention, the elastomer comprises silicon.

According to embodiments of the invention, the frame is bean-shaped.

According to embodiments of the invention, the cellularized structure further comprising at least one hardened synthetic material.

According to an aspect of the present invention, there is provided a cell perfusion device comprising:
a first sequence of at least three cell-culture wells; and
a second sequence of at least three cell-culture wells,
wherein each cell-culture well of the first sequence and each cell-culture well of the second sequence is directly connected to a sequential cell-culture well of the respective sequence allowing for fluid to flow through the first sequence of cell-culture wells in a sequential manner and allowing for fluid to flow through the second sequence of the cell-culture wells in a sequential manner; and
wherein a cell-culture well of the first sequence is not in fluid connection with a cell-culture well of the second sequence.

According to embodiments of the invention, each the cell-culture well of the first sequence and the second sequence is directly connected to the sequential cell-culture well of the respective sequence via at least one channel.

According to embodiments of the invention, the at least one channel comprises two channels.

According to embodiments of the invention, the two channels are parallel to one another and separated from one another by a distance of at least 200 microns.

According to embodiments of the invention, the two channels are configured to allow fluid to flow in a first direction in the first of the two channels and to flow in a second direction in the second of the two channels, the second direction being opposing to the first direction.

According to embodiments of the invention, each cell-culture well of the first and the second sequence comprises an inlet port configured to allow entry of the fluid and an outlet port configured to allow exit of the fluid.

According to embodiments of the invention, a first cell-culture well of the first and the second sequence comprises an inlet port configured to allow entry of the fluid and a last cell-culture well of the first and the second sequence comprises an outlet port configured to allow exit of the fluid.

According to embodiments of the invention, the inlet port and the outlet port are located at the same height from the bottom surface of each cell-culture well of the first or the second sequence and on opposing faces of each cell-culture well of the first or the second sequence.

According to embodiments of the invention, the first and the second sequence are organized as a row.

According to embodiments of the invention, the height is no more than 2 mm.

According to embodiments of the invention, the inlet port and the outlet port are of essentially the same dimensions.

According to embodiments of the invention, a diameter of the inlet port and the outlet port is between 0.01 to 1.5 mm.

According to embodiments of the invention, a bottom surface of the device is fabricated from glass.

According to embodiments of the invention, the glass is of a thickness of at least 0.1 mm.

According to embodiments of the invention, the cell perfusion device is a 384 well plate, a 96 well plate, a 24 well plate, a 12 well plate or a 6 well plate.

According to embodiments of the invention, the cell perfusion is a microfluidic device.

According to an aspect of the present invention, there is provided a system comprising:
the cell perfusion device disclosed herein; and
cellular matter, the cellular matter lining the channel.

According to embodiments of the invention, the cellular matter is selected from the group consisting of endothelial cells, pericytes, smooth muscles cells and astrocytes.

According to embodiments of the invention, each cell-culture well of the first sequence and the second sequence is seeded with biological cells.

According to embodiments of the invention, the biological cells form a 3-dimensional (3-D) cellular structure.

According to embodiments of the invention, each cell-culture well of the first and the second sequence comprises an inlet port configured to allow entry of the fluid and an outlet port configured to allow exit of the fluid.

According to an aspect of the present invention, there is provided a system comprising:
the cell perfusion device described herein;
wherein each cell-culture well of the first sequence and the second sequence is seeded with biological cells.

According to embodiments of the invention, each cell-culture well of the first and the second sequence comprises an inlet port configured to allow entry of the fluid and an outlet port configured to allow exit of the fluid.

According to embodiments of the invention, the biological cells form a 3-dimensional (3-D) cellular structure having an internal channel which allows perfusion of the 3D structure, wherein one end of the internal channel is connected to the inlet port and another end of the internal channel is connected to the outlet port.

According to embodiments of the invention, each cell-culture well of the first and/or the second sequence are seeded with an identical population of the biological cells.

According to embodiments of the invention, a first cell-culture well of the first sequence is seeded with a first population of biological cells and a second cell-culture well of the first sequence is seeded with a second population of biological cells, the first and the second population of biological cells being non-identical.

According to embodiments of the invention, the first population of biological cells comprises cancer cells and the second population of cells comprises lymph node cells.

According to embodiments of the invention, the first population of biological cells comprises cancer cells of a tumor and the second population of cells comprises metastasized cells of the tumor.

According to embodiments of the invention, the metastasized cells are selected from the group consisting of liver cells, lung cells, bone cells, and brain cells.

According to embodiments of the invention, the fluid comprises blood or a fraction thereof.

According to embodiments of the invention, the fraction comprises serum or plasma.

According to embodiments of the invention, the fraction comprises peripheral blood mononuclear cells (PBMCs) or buffy coat.

According to embodiments of the invention, walls of the internal channel are composed of cellular matter selected from the group consisting of endothelial cells, pericytes, smooth muscles cells, and astrocytes

According to embodiments of the invention, the 3D cellular structure is a tumor model comprising a plurality of cell types.

According to embodiments of the invention, at least a portion of the plurality of cell types are brain cells.

According to embodiments of the invention, the brain cells are selected from the group consisting of astrocytes, microglia cells, neuronal cells, and glioblastoma cells.

According to embodiments of the invention, at least a part of the cellular structure is generated by 3D printing.

According to embodiments of the invention, cells of the cellular structure are embedded in an extracellular matrix.

According to embodiments of the invention, the extracellular matrix comprises a synthetic material.

According to embodiments of the invention, the extracellular matrix is naturally occurring. According to embodiments of the invention, the walls of the channel are generated by 3D printing.

According to embodiments of the invention, the cell perfusion device is connected to a fluid source via a peristaltic pump or a micro-pump.

According to embodiments of the invention, the system further comprises at least one sensor for monitoring at least one parameter of the fluid.

According to embodiments of the invention, the parameter of the fluid is selected from the group consisting of oxygen concentration, glucose concentration, fatty acids composition, electrical resistance, pH, temperature, metabolite concentration, and flow rate.

According to embodiments of the invention, the at least one sensor is located at least one inlet and/or at least one outlet of at least one the cell culture well of the device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A: Drill jig. A jig that advances repetitive hole center location on multiple identical parts by acting as a template to direct the twist drill into the precise position of each proposed hole center.

FIG. 1B: Casting PDMS on an upside-down well plate. This will allow to create tight sealing between the 1.5 mm holes and the 25G needles.

FIG. 1C: A zoom-in on a casted formulation on top of a needle.

FIGS. 2A-C. Food dye perfusion through the 3D cancer model emphasizes the ability of an exemplary platform to be served as a pre-clinical tool for drug testing. Perfusion of an exemplary 3D-cancer vascularized array with A. Red food dye. B. UV dye. C. Blue food dye.

FIGS. 3A-D. Front view of four exemplary flow geometries, according to embodiments of the invention. A. Flow on top of the cells in 3D, B. Flow through the cells in 3D in one channel, C. Flow through the cells in 3D in two channels, possibly in two opposing directions, D. Flow through two channels from both sides of the cells in 3D.

FIG. 4A is an example of a printed silicon frame for use in a 3D printed lymph node.

FIG. 4B is a model of a cell fabricated 3D printed lymph node. In one embodiment, blue corresponds to B cells; green corresponds to dendritic cells; and yellow corresponds to macrophages. In another embodiment, blue corresponds to B cells, green corresponds to T cells; and yellow corresponds to dendritic cells.

FIG. 4C illustrates a method of sealing the 3D printed lymph node, according to embodiments of the invention.

FIG. 4D illustrates an exemplary design of a frame which can be used to 3D print a lymph node.

FIG. 5 is an image of a 3D-bioprinted fibrin-gelatin lymph node showing embedded areas of B cells (cyan), dendritic cells (green) and macrophages (red).

FIG. 6A is a schematic illustration of an exemplary cell perfusion system comprising peristatic pumps, media reservoir, waste reservoir and filters, according to embodiments of the invention. A representative blow-out image of a well that includes a 3D-bioprinted tumor consisting of GFP-labeled cancer cells and mCherry-endothelial cells. All other microenvironment stromal cells are not labeled but are included and support tumor growth and sprouting.

FIG. 6B is a magnification of the cell perfusion system illustrated in FIG. 6A.

FIGS. 6C-6D illustrate exemplary sequences of cell culture wells according to embodiments of the invention.

FIG. 7 is a bar graph illustrating cell viability % of mucosal melanoma patient-derived cells which were treated with 16 different treatments as mono-therapies or combination therapies.

### DESCRIPTION OF SPECIFIC EMBODIMENTS OF THE INVENTION

The present invention, in some embodiments thereof, relates to cell perfusion systems having a multi-well platform for use in carrying out perfusion studies.

The cell culture wells of the device are organized as sequences, wherein a well of the sequence is directly connected to the sequential well of that sequence (e.g. via channels or ports) allowing fluid to flow from the first to the last well of the sequence in a sequential manner. Each sequence is independent of other sequences in the device. Accordingly, fluid cannot flow from one sequence to the next. The device is preferably configured such that there is no fluid connection between one sequence to another sequence.

Each well of a single sequence can be seeded with a population of cells. In one scenario, the population of cells (and culturing conditions) are identical for each well of a sequence. In this configuration, each sequence serves as an independent study having a number of repeats according to the number of wells in the sequence. Each independent sequence in the device may be perfused with different compounds (e.g. drugs, chemicals, growth factors, mRNA/CRISPR/Cas9/RNAi nanoparticles). Alternatively, each sequence in the device may be perfused with the same compound, but at different concentrations (mimicking serial doses). In another scenario, the population of cells are non-identical in wells of a single sequence. Thus, for example, a first well of a sequence may be seeded with a first population of cells and the second well of the sequence is seeded with a second population of cells. The perfusion medium may be supplemented with a compound (as detailed above). The first population of cells releases a factor into the perfusion medium in response to the compound. The factor then flows to the next well of the sequence and contacts the second population of cells seeded therein. This arrangement allows mimicking of sequential biological processes, including, for example, tumor metastasis (e.g. the first well of the sequence may comprise cells which form a 3D structure resembling a tumor, and the second well of the sequence may comprise cells which form a lymph node).

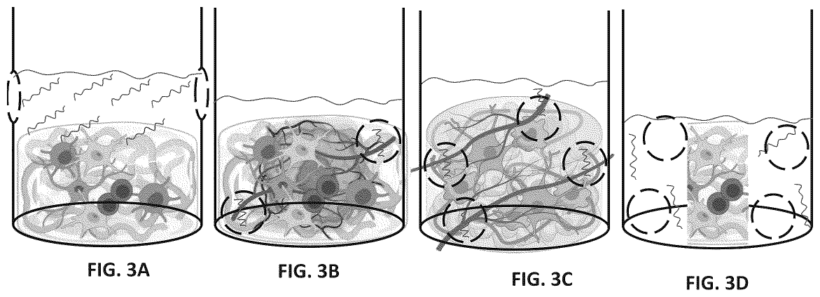

Channels which connect wells of a sequence may be lined with cells (e.g. endothelial cells, smooth muscle cells and/or pericytes) such that the channels may be considered a vasculature connecting two cell populations seeded in sequential wells of a sequence. The channels may be fully enclosed (e.g. hollow tube) or may be open channel (e.g. a ridge). The channel may be in direct contact (e.g. traverse) with the cellular material seeded on the wells (see for examples FIGS. 3B and 3C) or may traverse the well without being in direct contact with the cellular material (see for examples FIGS. 3A and 3D).

Alternatively, the 3D structure of cells comprised in the wells may include at least one channel (e.g. a central channel). In one embodiment, the cells seeded in the wells comprise two channels (see FIG. 3C). Optionally, the channel(s) may be lined with or composed of endothelial cells, pericytes, and/or smooth muscle cells. The channel may connect between an inlet port and an outlet port of a single well. Thus, fluid may flow directly from one 3D structure in one well to another 3D structure in a sequential well via the central channel in a single direction. In the embodiment, where the cellular structure comprises two channels, the flow in each of the channels can be opposing.

Exemplary methods for forming channels and 3D structures are summarized in the Examples section, herein below.

Thus, according to a first aspect of the invention, there is provided a cell-perfusion system comprising:
a sequence of at least two cell-culture wells comprising:
   a first cell-culture well which comprises a first population of cells; and
   a second cell-culture well which comprises a second, non-identical population of cells,
   a fluid pathway interconnecting the first cell-culture well and the second cell-culture well so as to allow for fluid to flow through the sequence in a sequential manner.

The term “cell perfusion system” refers to a system which allows biological cells to be cultured, while continuously exchanging culture medium. Fresh medium replenishes nutrients and carbon sources, while cellular waste and medium depleted of nutrients are removed.

At its minimum the system comprises a sequence of at least two cell-culture wells which are connected to one another such that fluid flows from the first well to the second well. In all embodiments, the flow may be uni-directional or bi-directional. Preferably, the flow is uni-directional.

In another embodiment, the sequence comprises at least three cell culture wells, which are connected to one another such that fluid flows from the first well to the second well and from the second well to the third well. Fluid does not flow directly from the first well to the third well (i.e. without passing through the third well).

It will be appreciated that the sequence may comprise any number of wells, 2, 3, 4, 5, 6, 7, 8, 9, 10, 11, 12, or more. In one embodiment, the sequence comprises at least three wells.

The wells of a sequence may be organized in the system randomly or according to rows.

The cell culture well may be the size of a well of a standard 384-well plate or 96-well plate or a 24-well plate or 12-well plate or 6-well plate, as known in the art.

The cell perfusion system may comprise more than one sequence of connected cell culture wells.

According to a particular embodiment, the cell perfusion system comprises at least two sequences. In this embodiment, each cell-culture well of the first sequence and each cell-culture well of the second sequence is directly connected to a sequential cell-culture well of the respective sequence allowing for fluid to flow through the first sequence of cell-culture wells in a sequential manner and allowing for fluid to flow through the second sequence of the cell-culture wells in a sequential manner. The cell-culture well of the first sequence is not in fluid connection with a cell-culture well of the second sequence.

It will be appreciated that the cell perfusion system may comprise additional sequences—e.g., 3, 4, 5, 6, 7, 8, 9, 10 or more, each sequence being configured such that fluid flows independently through the sequence, as described herein above. Wells of each sequence may be organized in rows, and each sequence may be located at a predetermined position on the cell perfusion system (e.g. as columns).

The choice of materials for fabricating the cell perfusion system typically depends upon the particular material properties (e.g., specific tissue mechanical properties and composition, solvent resistance, stiffness, gas permeability, and/or temperature stability) required for the application being conducted. The culture wells should be fabricated from a material suitable for culturing cells.

Additional details regarding the type of materials that can be used in the manufacture of the components of the system disclosed herein are set forth in Unger et al. (2000) Science 288:113-116, and PCT Publications WO 02/43615, and WO 01/01025. Exemplary low-background substrates include those disclosed by Cassin et al., U.S. Pat. No. 5,910,287 and Pham et al., U.S. Pat. No. 6,063,338.

Preferred elastomers of the instant invention are biocompatible, gas permeable, optically clear elastomers useful in soft lithography including silicone rubbers, most preferably PDMS. Other possible elastomers for use in the devices of the invention include, but are not limited to, polyisoprene, polybutadiene, polychloroprene, polyisobutylene, poly(styrene-butadiene-styrene), the polyurethanes, and silicone polymers; or poly(bis(fluoroalkoxy)phosphazene) (PNF, Eypel-F), poly(carborane-siloxanes) (Dexsil), poly(acrylonitrile-butadiene) (nitrile rubber), poly(1-butene), poly(chlorotrifluoroethylene-vinylidene fluoride) copolymers (Kel-F), poly(ethyl vinyl ether), poly(vinylidene fluoride), poly(vinylidene fluoride-hexafluoropropylene) copolymer (Viton), elastomeric compositions of polyvinylchloride (PVC), polysulfone, polycarbonate, polymethylmethacrylate (PMMA), and polytertrafluoroethylene (Teflon).

In one embodiment, the cell perfusion system is fabricated from a material including polystyrene, polycarbonate, cyclo-olefins (polypropylene), glass or quartz.

In one embodiment, the cell perfusion system comprises a multi-well plate which is adapted to include a fluid pathway which interconnects the first cell culture well to the second cell culture well. Multi-well plates are well known to those skilled in the art (e.g. 384-well plate or 96-well plate or a 24-well plate or 12-well plate or 6-well plate).

Exemplary multi-well plates include BD Falcon™ multi-well plates, available in 24-well plates and 96-well plates.

The plates may be injection molded and may be disposable or reusable. The plates may be sterilized using any suitable sterilization method known to those skilled in the art.

According to a particular embodiment, the bottom of the cell culture plate is fabricated from a transparent material such as glass (e.g. having a thickness of at least 0.1 mm), which allows for imaging of cells present cultured in the plates.

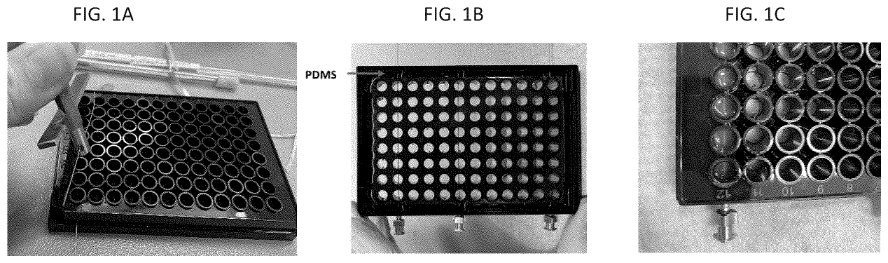

The fluid pathway which connects the well of the sequence may be a channel, a hole in the wall of the plate or any other connecting means. In one embodiment, the fluid pathway comprises a single channel connecting between wells. The channel may be flexible (e.g. fabricated from tubing) or rigid. In an exemplary embodiment, the channel is molded into the plate. An exemplary method of creating a channel through a cell culture plate is illustrated in FIG. 1A and described in Example 1 herein below.

In another embodiment, the fluid pathway comprises two channels which are parallel to one another and separated from one another by a distance of at least 200 microns, at least 300 microns, at least 500 microns, at least 1000 microns or more. The fluid pathway may be configured such that fluid flows in a first direction in the first of the two channels and flows in a second direction (e.g. opposite direction) in the second of the two channels.

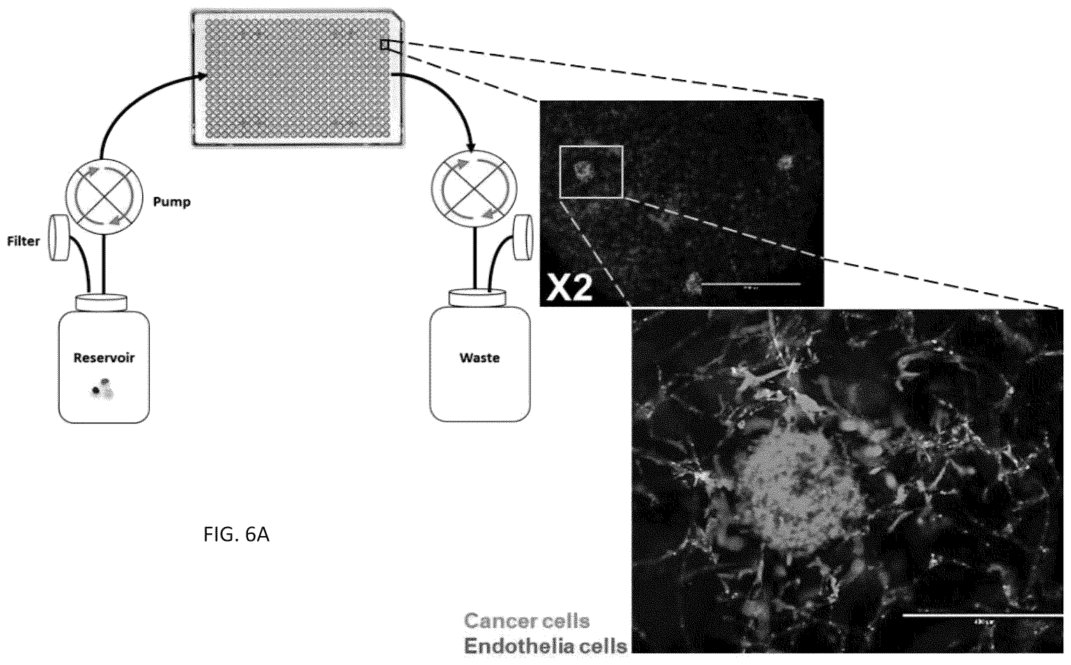

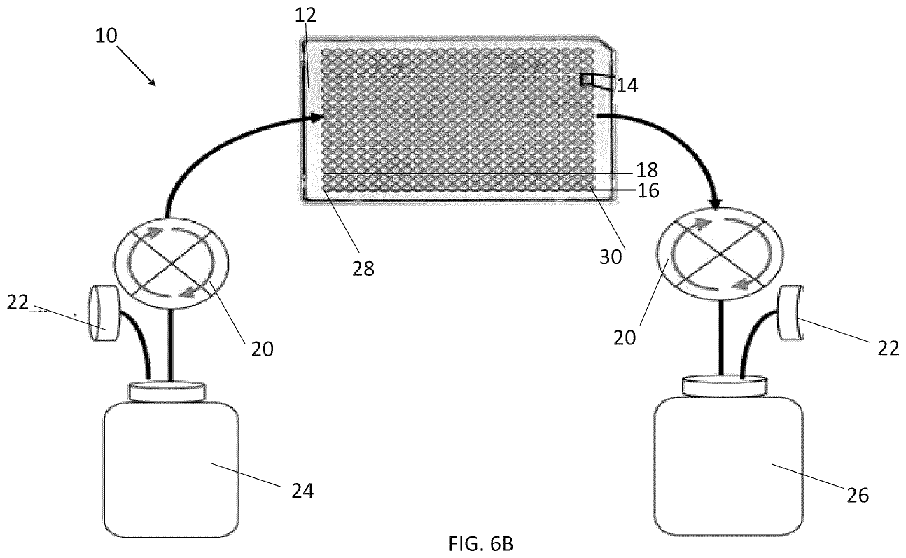

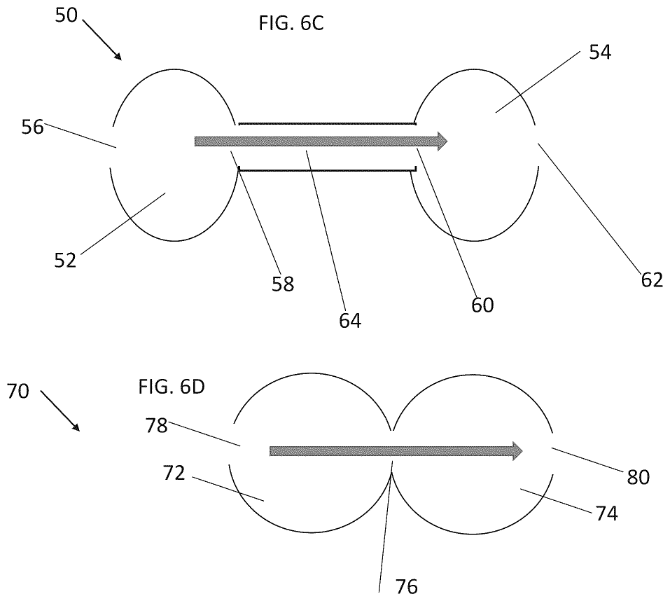

Each cell-culture well of the sequence/sequences comprises an inlet port configured to allow entry of the fluid and an outlet port configured to allow exit of the fluid. In one embodiment, the inlet port of the first cell culture well of each sequence allows entry of fluid from a fluid reservoir and the outlet port of the last cell culture well of each sequence allows exit of the fluid to a waste reservoir. The entry fluid reservoir and the waste fluid reservoir may be identical (i.e. may be continually replenished) or separate (as in FIG. 6A-B).

In one embodiment, the inlet port and the outlet port are located at the same height (e.g. no more than 1 mm, no more than 2 mm, no more than 3 mm) from the bottom surface of each cell-culture well and on opposing faces of each cell-culture well of any one sequence.

In another embodiment, the inlet port and the outlet port of each cell culture well are essentially of the same dimensions. In still another embodiment, the inlet port and the outlet port of each cell culture well of a particular sequence are essentially of the same dimensions (e.g. having a diameter between 0.01 to 1.5 mm).

Fluids may be passively or actively infused into the cell perfusion systems such as by capillary forces or pumps (e.g., external pumps, e.g., peristaltic pumps or electro-osmotically pumps, gravitational flow).

Liquid flow through the perfusion system may be regulated using a valve.

A “valve” is a component of a device that regulates flow through the system by substantially inhibiting (or reducing) flow upon closure. Substantially inhibiting the flow means that flow is inhibited at least 80%, preferably at least 90%, more preferably at least 95%, even more preferably at least 99%, most preferably flow is completely (i.e., 100%) inhibited. The size of the valve is dependent on the size and shape of the channel and the amount of pressure required to close the valve.

Exemplary sequences of cell cultures wells are depicted in FIGS. 6C and 6D. FIG. 6C illustrates sequence 50 which comprises a first culture well 52 and a second culture well 54 which is consecutive in the series 50. Fluid flows in the direction of the blue arrow through entry port 56 into first culture well 52, into channel 64 via exit port 58 and subsequently into second culture well 54 via entry port 60. Fluid exits second culture well via exit port 62.

FIG. 6D illustrates sequence 70 which comprises a first culture well 72 and a second culture well 74 which is consecutive in the series 70. Fluid flows in the direction of the blue arrow through into first culture well 72 via inlet 78, into second culture well 74 via hole 76 and out second culture well 74 via outlet 80.

An exemplary cell perfusion system contemplated by the present invention is presented in FIG. 6B. Perfusion system 10 includes plate 12 which comprises a plurality of cell culture wells 14. The cell culture wells are organized in rows. A fluid path connects cell culture wells to generate a sequence of interconnected wells (e.g. 16, 18). In an exemplary embodiment, fluid flows from fluid reservoir 24 via pump 20 via a tube and enters the first well 28 of sequence 16. Fluid continues to flow through sequence 16 until it reaches the last well 30 of sequence 16. From well 30, fluid is pumped out of the plate via pump 20 and enters waste reservoir 26. Air enters the system via filter 22. For the sake of convenience, only two sequences are illustrated, although it will be appreciated that the system can utilize any number of sequences of interconnected wells.

Additional examples of cell perfusion systems contemplated by the present invention include those presented in U.S. Pat. No. 11,154,858B2, U.S. Pat. No. 20,210,284944A1 and EP3892713A1, the contents of which are incorporated herein.

In one embodiment, the cell perfusion system comprises a microfluidic device.

As used herein the phrase “microfluidic device” refers to a synthetic device in which minute volumes of fluids are flowed. The flow channel is generally fabricated at the micron to sub-micron scale, e.g., the channel typically has at least one cross-sectional dimension in the range of less than about 1 mm.

As mentioned, the cell perfusion system described herein is for culturing cellular material. In one embodiment, the first well of a sequence is seeded with a first population of cells and the second well of the sequence is seeded with a second population of cells.

In some embodiments, any vertebrate cell is suitable for use in the cell perfusion system. In further embodiments, the cells are, by way of non-limiting examples, contractile or muscle cells (e.g., skeletal muscle cells, cardiomyocytes, smooth muscle cells, and myoblasts), connective tissue cells (e.g., bone cells, cartilage cells, fibroblasts, and cells differentiating into bone-forming cells, chondrocytes, or lymph tissues), bone marrow cells, endothelial cells, skin cells, epithelial cells, breast cells, vascular cells, blood cells, lymph cells, neural cells, Schwann cells, gastrointestinal cells, liver cells, pancreatic cells, lung cells, tracheal cells, corneal cells, genitourinary cells, kidney cells, reproductive cells, adipose cells, parenchymal cells, pericytes, mesothelial cells, stromal cells, undifferentiated cells (e.g., embryonic cells, stem cells, and progenitor cells), endoderm-derived cells, mesoderm-derived cells, ectoderm-derived cells, and combinations thereof.

According to a particular embodiment, the cells are intact (i.e., whole), and viable.

The cells may be primary cells, immortalized cells or derived from cell lines.

The cells may be fresh, frozen or preserved in any other way known in the art (e.g., cryopreserved).

In one embodiment, the cells used in the microfluidic system are genetically modified (e.g. to express a therapeutic agent or a detectable moiety) by any suitable method known in the art.

According to some embodiments, the cells or parts of the cell types are labeled.

Methods of labeling cells and useful dyes and genetic dyes are well known in the art.

Cells, cell clusters, organelles visualization with selective stains or dyes or fluorescent proteins are key tools in fluorescence imaging of cells and tissues. These specific stains are suitable counterstains to antibodies to help the identification of location-specific targets of interest within the cell. Dyes for live cell staining of organelles are available in a broad spectrum of colors. Such labels are known in the art.

Examples include but are not limited to those listed in Salipalli et al. 2014 BMC Cell Biol. 15:26, which is hereby incorporated by reference in its entirety.

Measures are taken for using different labels for different cell types according to the intended use.

According to other embodiments, the cells or parts pf the cell types are unlabeled.

In some embodiments, the cells are IPS-based and then differentiated and in other embodiments, the cells are adult, differentiated cells. In further embodiments. “differentiated cells” are cells with a tissue-specific phenotype consistent with. for example, a muscle cell, a fibroblast, or an endothelial cell at the time of isolation, wherein tissue-specific phenotype (or the potential to display the phenotype) is maintained from the time of isolation to the time of use. In other embodiments, the cells are adult, non-differentiated cells. In further embodiments, “non-differentiated cells” are cells that do not have, or have lost, the definitive tissue-specific traits of for example, muscle cells, fibroblasts, or endothelial cells. In some embodiments, non-differentiated cells include stem cells. In further embodiments, “stem cells” are cells that exhibit potency and self-renewal. Stem cells include, but are not limited to, totipotent cells, pluripotent cells, multipotent cells, oligopotent cells, unipotent cells, and progenitor cells. In various embodiments, stem cells are embryonic stem cells, adult stem cells, amniotic stem cells, and induced pluripotent stem cells. In other embodiments, the cells are a mixture of adult, differentiated cells and adult, non-differentiated cells.

According to a particular embodiment, the cells are derived from (or comprise) stem cells—e.g., adult stem cells such as mesenchymal stem cells or pluripotent stem cells such as embryonic stem cells or induced pluripotent stem cells (iPSCs). The stem cells may be modified so as to undergo ex vivo differentiation prior to seeding on the culture plates or may be used as pluripotent stem cells and further differentiated in situ.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (sec WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

It will be appreciated that commercially available stem cells can also be used according to some embodiments of the invention. Human ES cells can be purchased from the NIH human embryonic stem cells registry [www (dot) grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CTI, CT2, CT3, CT4, MA135, Encavour-2, WIBRI, WIBR2, WIBR3, WIBR4, WIBR5, WIBR6, HUES 45, Shef 3, Shef 6, BJNhem19, BJNhem20, SA001, SA001.

Induced pluripotent stem cells (iPSCs; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as omentum) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc/1-Myc in omental cells.

In one embodiment, the cells are diseased cells—e.g. tumor/cancer cells. In one embodiment, the cells may be derived from a diseased subject (mammalian subject, e.g. human). In another embodiment, the cells may be derived from a healthy subject.

The term “cancer” as used herein refers to an uncontrolled, abnormal growth of a host's own cells which may lead to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood-forming tissue (e.g., bone marrow tissue); lymphomas and myelomas which are cancers of immune cells; and central nervous system cancers which include cancers from brain and spinal tissue. “Cancer(s),” “neoplasm(s),” and “tumor(s)” are used herein interchangeably. As used herein, “cancer” refers to all types of cancer or neoplasm or malignant tumors, including leukemias, carcinomas, and sarcomas, whether new or recurring.

Specific examples of cancer cells which may be used in the perfusion system described herein include cells of, but are not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; triple negative breast cancer, Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic;

leukemia, acute myeloid; leukemia, acute myeloid, with cosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, malignant melanoma, meningioma; multiple endocrine neoplasia; multiple myeloma, myeloid malignancy, predisposition to; myxosarcoma, neuroblastoma; osteosarcoma; osteocarcinoma, ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, basal cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

According to a particular embodiment, the cells are selected from the group consisting of breast cancer cells, melanoma cells, colorectal cancer cells, lung cancer cells, pancreatic ductal adenocarcinoma (PDAC) cells, gastric cancer cells, ovarian cancer cells and brain cancer cells (e.g. glioblastoma cells).

According to another embodiment, the cells comprise melanoma cells.

Malignant melanomas are clinically recognized based on the ABCD (E) system, where A stands for asymmetry, B for border irregularity, C for color variation, D for diameter >5 mm, and E for evolving. Further, an excision biopsy can be performed in order to corroborate a diagnosis using microscopic evaluation. Infiltrative malignant melanoma is traditionally divided into four principal histopathological subgroups: superficial spreading melanoma (SSM), nodular malignant melanoma (NMM), lentigo maligna melanoma (LMM), and acral lentiginous melanoma (ALM). Other rare types also exists, such as desmoplastic malignant melanoma. A substantial subset of malignant melanomas appear to arise from melanocytic nevi and features of dysplastic nevi are often found in the vicinity of infiltrative melanomas. Melanoma is thought to arise through stages of progression from normal melanocytes or nevus cells through a dysplastic nevus stage and further to an in situ stage before becoming invasive. Some of the subtypes evolve through different phases of tumor progression, which are called radial growth phase (RGP) and vertical growth phase (VGP).

In another exemplary embodiment, at least one of the wells comprise cells of an organ model.

The organ model comprises a plurality of cells (e.g. at least two cell types, at least three cell types, at least four cell types) which interact together and carry out at least one biological function of the organ. In one embodiment, the healthy organ is vascularized. In one embodiment, the organ is bioprinted. In another embodiment, the organ is commercially available e.g. Elvesys, Mimetas, Mesobiotech, Emulate or Insphero. Examples of organs-on-a-chip include liver, lung, heart, kidneys, brain.

In one embodiment, the first population of cells cultured in a cell culture well of the system is identical to the second population of cells cultured in a consecutive cell culture well of the system.

As used herein the term “identical” with respect to cellular populations refers to cells of the same cell type. In one embodiment, cells of the same type are derived from the same subject. In another embodiment, cells of the same type are derived from different subjects.

In one embodiment, cells of a cell type expresses a unique cell marker and can be distinguished from other cells in the cell environment by virtue of expression of the unique cell marker or a unique repertoire of cell markers.

In one embodiment, the unique cell marker is a cell surface marker.

The surface cell target may include a carbohydrate, lipid, protein, extracellular protein, cell surface protein, B cell receptor, T cell receptor, major histocompatibility complex, tumor antigen, receptor, intracellular protein, or any combination thereof. In some embodiments of the present invention, surface cell targets include CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CDw12, CD13, CD14, CD15u, CD15s, CD15su, CD16b, CD17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45 RA-CD 45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62 5262 62L, CD, P, CD, CD64, CD65s, CD66a, CD66b, CD66c, CD66d, CD66e, CD66f, CD68, CD69, CD70, CD71, CD72, CD73, CD74, CD75s, CD77, CD79a, CD79b, CD80, CD81, CD82, CD83, CD84, CD85a, CD85d, CD85j, CD85k, CD86, CD87, CD88, CD89 CD45RB, CD45RC, CD45RO, CD46, CD47, CD48, CD49a, CD49b, CD49c, CD49d, CD49c, CD49f, CD50, CD51, CD52, CD53, CD54, CD55, CD56, CD57, CD58, CD59, CD60a, CD60b, CD60c, CD61, CD62E, CD L, CD P, CD, CD64, CD65s CD66a, CD66b, CD66c, CD66d, CD66e, CD66f, CD68, CD69, CD70, CD71, CD72, CD73, CD74, CD75s, CD77, CD79a, CD79b, CD80, CDS1, CD82, CD83, CD84, CDS5a, CD85d, CD85j, CDS5k, CD86, CD87, CD88, CD89, CD80, CD201, CD202b, CD203c, CD204, CD205, CD206, CD207, CD208, CD209, CD210, CDw210b, CD212, CD213a1, CD213a2, CD215, CD217a, CD218b, CD220, CD221, CD222, CD223, CD224, CD225, CD226, CD227, CD228, CD229, CD230, CD231, CD232, CD233, CD234, CD235a, CD235b, CD236R, CD, CD239, CD240CE, CD240DCE, CD240D, CD, CD242, CD243, CD244, CD245, CD246, CD247, CD248, CD249, CD252, CD253, CD254, CD256, CD266, CD267, CD268, CD269, CD270, CD223, CD273, CD274, CD275, CD276, CD277, CD279, CD283, CD280, CD281, CD282, CD289, CD2, CD276, CD 277; CD 293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD308, CD309, CD312, CD314, CD315, CD316, CD317, CD318, CD319, CD320, CD32, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, CD363, CD365, CD366, CD367, CD368, CD369, CD370, CD371, BCMA, protein, beta-2.

In various embodiments, the first population of cells cultured in a cell culture well of the system is non-identical to the second population of cells cultured in a consecutive cell culture well of the system.

As used herein the term “non-identical” with respect to cellular populations refers to cells of different cell types. In one embodiment, cells of different cell types are derived from the same subject. In another embodiment, cells of different cell types are derived from different subjects.

In one embodiment, cells of different cell type expresses non-identical unique cell markers and can be distinguished from each other by virtue of expression of the unique cell marker or a unique repertoire of cell markers.

In still another embodiment, cells of different cell types are tumor cells derived from different locations in the body of a single subject—i.e. primary tumor cells and metastasized cells of the tumor.

According to a particular embodiment, the first population of cells comprise primary tumor cells and the second population of cells comprise lymph node cells. The lymph node cells may be part of a 3D structure (e.g. as described herein below) and comprise cells derived from the same subject as the primary tumor cells are derived. Optionally, the 3D structure further comprises metastasized cells of the primary tumor. In another embodiment, the first population of cells comprise primary tumor cells and the second population of cells comprise cells of a tissue to which the primary tumor cells have metastasized. Optionally, the cells of the tissue further comprise cells of the metastasized tumor. In still another embodiment, the first population of cells comprise lymph node cells. The lymph node cells may be part of a 3D structure (e.g. as described herein below) and may comprise tumor cells of a metastasized tumor. The second population of cells comprise cells of a tissue to which a primary tumor cells have metastasized. Optionally, the cells of the tissue further comprise cells of the metastasized tumor.

As used herein “brain metastasis cell” refers to a cancer cell that metastasized to the brain however its primary origin is not brain. Metastatic brain tumors (i.e., cancer that began somewhere else in the body spreads to the brain and causes a mass or brain tumor) include, but are not limited to, lung, breast, melanoma, colon, kidney and thyroid gland cancers.

Table 1 herein below provides examples of different pairs of cell populations contemplated by the present inventors.

TABLE 1

| First cell population | Second cell population |
|---|---|
| Primary melanoma cells | Metastasized melanoma cells |
| | in the lymph node (TDLN- |
| | tumor-draining lymph node) |
| Primary melanoma cells | Metastasized melanoma cells |
| | in the brain |
| Primary breast cancer cells | Metastasized breast cancer |
| | cells in the lymph node |
| Primary breast cancer cells | Metastasized breast cancer |
| | cells in the brain |
| Primary colorectal cancer | Metastasized CRC cells in the |
| (CRC) cells | lymph node |
| Primary CRC cells | Metastasized CRC cells in the |
| | brain |
| Primary lung cancer (e.g. | Metastasized lung cells in the |
| NSCLC) cells | lymph node |
| Primary lung cancer cells | Metastasized lung cancer in |
| | the brain |

In some embodiments, the first cell type in a first culture well of a series comprises primary tumor cells, the second cell type in a second culture well of the series comprises lymph node cells and the third cell type in the third culture well of the series comprises cells of a secondary tumor (i.e. cells which have metastasized to another organ.

In still other embodiments, at least one of the wells of the series comprises primary or secondary tumors and the subsequent well of the series comprises cells of a healthy organ model (e.g. organ on a chip).

In some embodiments, one or more specific cell types are derived from two or more distinct human donors. In some embodiments, one or more specific cell types are derived from a particular vertebrate subject. In further embodiments, one or more specific cell types are derived from a particular mammalian subject. In still further embodiments, one or more specific cell types are derived from a particular human subject.

The present inventors contemplate that at least one cell population cultured in the perfusion system described herein forms or is formed in a 3D cellular structure.

In one embodiment, the 3D cellular structure comprise cells of a tumor (e.g. a tumor model). In another embodiment, the 3D cellular structure comprises cells of a lymph node. The 3D cellular structure may be obtained by biopsy from a patient or may be generated—e.g. by 3D printing. Each of these 3D cellular structures will be described herein below.

Pericytes and endothelial cells and smooth muscle cells may be used establish the vascularization in a 3D cellular structure according to some embodiments of the invention.

As used herein “pericytes” refer to cells present at intervals along the walls of capillaries (and post-capillary venules). In the central nervous system (CNS), they are important for blood vessel formation, maintenance of the blood-brain barrier, regulation of immune cell entry to the central nervous system (CNS) and control of brain blood flow. Typical markers include, but are not limited to α-SMA and NG2.

Examples of normal human pericyte cell lines and primary cells include but are not limited to Human Brain Vascular Pericytes (HBVP) Catalog #1200, ScienCell.

The Examples section which follows provides examples of cell lines of pericytes, endothelial and microglial cell from commercial vendors such as the ATCC and ScienCell (California, USA).

As used herein “endothelial cells” to the cells which line the interior surface of the blood vessel. Endothelial cells release substances that control vascular relaxation and contraction as well as enzymes that control blood clotting, immune function and platelet (a colorless substance in the blood) adhesion. Typical markers include, but are not limited to, CD31/PECAM-1. Angiotensin-converting enzyme, Factor VIII-related antigen, Ulex europaeus I agglutinin binding/O (H) blood-type antigen, Vascular endothelial cadherin, CD36, CD105/endoglin, CD73 and AAMP.

Examples of human endothelial cells cell lines and primary cells include but are not limited to HAUC, HPAEC, HUVEC, HDMVEC, HAMEC, HCMEC/D3 and HBEC, all available from ATCC.

It will be appreciated that cells as described herein are commercially available or can be self-isolated.

It will be appreciated that the use of more than one marker and/or the use of negative markers may improve the specificity of identification of the desired cells. The use of more than one marker is also referred to as a “signature”.

### 3D Tumor

According to a particular embodiment, the 3D structure is a tumor model. A tumor model typically comprises malignant cells, and non-malignant cells, i.e., stroma and vasculature such that the tumor model represents also the tumor microenvironment.

According to a specific embodiment, the cells of the 3D tumor model can be of primary cells, non-immortalized cells, freshly isolated from a patient without any culturing or cloning, cell lines or a combination of same.

According to a specific embodiment, the cells of the 3D tumor model are mammalian cells e.g., mouse.

According to a specific embodiment, the cells of the 3D tumor model are human cells. According to some embodiment, the cells of the 3D tumor model are from a single host (e.g., at least the tumor cells).

According to a specific embodiment, the cells of the 3D tumor model are from different hosts.

According to a specific embodiment, the cells of the 3D tumor model are from different hosts (e.g., different human beings) or organism origin e.g., human and mouse.

According to a specific embodiment, each of the plurality of cell types of the 3D tumor model is from different organisms (e.g., different patients).

According to a specific embodiment, some of the plurality of cell types are from different organisms (e.g., different patients, e.g., the tumor cells and stroma are from one organism and the vascularization cells are from other(s)).

According to a specific embodiment, each of the plurality of cell types is from the same organism.

According to a specific embodiment, all the cells are autologous to the subject on which personalized screening for drugs will take place using the 3D model (e.g., full HLA matchability as described below).

As used herein “full match HLA” refers to 100% identical HLA alleles. Embodiments of the invention relate to 3D tumor models which comprise cells derived from a single donor.

According to some embodiments, these tumors are micro-engineered based on in vivo imaging data, and their 3D structure features a high match to their original architecture.

Such 3D tumor models may find various uses in drug screening and personalized therapy. For example, several drugs (as monotherapy or combination therapies) can be tested on them within days-a process that is useful in cases of aggressive tumors.

According to a specific embodiment, at least some of the cell types are comprise fully matched HLA.

Generally, in order to produce the model, the tumor cells and stroma (microglia +astrocytes) are retrieved and mixed with a first synthetic material forming the tumor bio-ink.

When obtained from tissue biopsies the tissue is mechanically and/or enzymatically processed to obtain viable single cells or aggregates thereof not exceeding 500 (e.g., 250, 100, 50 or 10) cells/aggregate. An exemplary protocol for isolation is provided in the Examples section which follows. Typically, a tumor biopsy is obtained. The tissue is processed to obtain cells in suspension which are selected by marker expression. Vascular forming cells such as the pericytes and endothelial cells are isolated and the rest of the cells are taken to form the tumor bio-ink. Of note, the tumor bio-ink may comprise the tumor cells, microglia and astrocytes but other cells can be present too in this suspension.

An exemplary method for producing a glioblastoma tumor model is provided in WO2023/007500, the contents of which are incorporated herein by reference.

Typically cells are dispensed in a configured pattern that corresponds to a desired shape of respective three-dimensional object or a part thereof. According to some embodiments, dispensing can be performed digitally, for example, via any of the known additive manufacturing methodologies as described herein, e.g., printing, bioprinting, or via non-digital methodologies such as, for example, casting (e.g., mold casting).

As used herein in the context of the present embodiments, the term “casting” refers to a methodology in which a liquid material or formulation is placed in a mold and allows to harden, to thereby form an object, or a part thereof, in a shape of the mold.

As used herein, “bioprinting” or it's shortened version “printing” means practicing an additive manufacturing process, preferably a 3D-inkjet printing process or extrusion printing, while utilizing one or more bio-ink formulation(s) that comprises cells or cellular components (e.g., cell solutions, cell-containing gels, cell suspensions, cell concentrations, multicellular aggregates, multicellular bodies, etc.) via methodology that is compatible with an automated or semi-automated, computer-aided, additive manufacturing system as described herein (e.g., a bioprinter or a bioprinting system).

### Additive Manufacturing (AM):

According to some embodiments of this aspect, the method is effected by sequentially forming a plurality of layers in a configured pattern corresponding to the shape of the object, thereby forming the object. According to some embodiments of this aspect, formation of each layer is effected by dispensing at least one uncured building material, and exposing the dispensed building material to a curing condition to thereby form a hardened (cured) material.

Herein throughout, the phrase “uncured building material” or “uncured building material formulation” collectively describes the materials that are used to sequentially form the layers, as described herein. This phrase encompasses uncured materials which form the final object, namely, one or more uncured modeling material formulation(s), and optionally also uncured materials used to form a support, namely uncured support material formulations. According to some embodiments, this refers to the first synthetic material and/or second synthetic material in their curable/uncured form.

An uncured building material can comprise one or more modeling formulations, and can be dispensed such that different parts of the object are made upon curing different modeling formulations, and hence are made of different cured modeling materials or different mixtures of cured modeling materials.

The method of the present embodiments manufactures three-dimensional objects in a layer-wise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different cell type). Thus, different target locations can be occupied by different building materials (e.g., a modeling formulation and/or a support formulation, as defined herein).

The final three-dimensional object is made of the modeling material or a combination of modeling materials or a combination of modeling material/s and support material/s or modification thereof (e.g., following curing). All these operations are well-known to those skilled in the art of additive manufacturing (also known as solid freeform fabrication).

In some exemplary embodiments of the invention, an object is manufactured by dispensing a building material that comprises two or more different modeling material formulations, each modeling material formulation from a different dispensing head of the AM apparatus. The modeling material formulations are, optionally and preferably, deposited in layers during the same pass of the printing heads. The modeling material formulations and/or combination of formulations within the layer are selected according to the desired properties of the object.

An exemplary 3D printing method according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD).

The method continues by dispensing droplets of the uncured building material as described herein in layers, on a receiving medium, using one or more printing heads, according to the printing data. The receiving medium can be a tray of a printing system or a previously deposited layer.

Once the uncured building material is dispensed on the receiving medium according to the 3D printing data, the method optionally and preferably continues by exposing the deposited layers to a curing condition. Preferably, the curing condition is applied to each individual layer following the deposition of the layer and prior to the deposition of the previous layer.

Exposure to a curing condition is typically performed using a curing energy source which can be, for example, a radiation source, such as an ultraviolet or visible or infrared lamp, or other sources of electromagnetic radiation, or electron beam source, depending on the modeling material formulation(s) being used. The curing energy source serves for curing or solidifying (hardening) at least the modeling material formulation(s). Alternatively, a curing condition can include a presence of a chemical or biological reagent that promotes curing (e.g., an enzyme or a catalyst).

Some AM processes according to the present embodiments involve dispensing materials (e.g., hydrogels, for example, Pluronic hydrogels) without exposing these materials to curing energy but rather to a curing condition as defined herein. Such hydrogels can harden, for example, in the presence of calcium ions or when a formulation containing same is cooled.

Some embodiments contemplate the fabrication of an object by dispensing different formulations from different dispensing heads. These embodiments provide, inter alia, the ability to select formulations from a given number of formulations and define desired combinations of the selected formulations and their properties. According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object. A printing system utilized in additive manufacturing may include a receiving medium and one or more printing heads. The receiving medium can be, for example, a fabrication tray that may include a horizontal surface to carry the material dispensed from the printing head. The printing head may be, for example, an ink jet head having a plurality of dispensing nozzles arranged in an array of one or more rows along the longitudinal axis of the printing head. The printing head may be located such that its longitudinal axis is substantially parallel to the indexing direction. The printing system may further include a controller, such as a microprocessor to control the printing process, including the movement of the printing head according to a pre-defined scanning plan (e.g., a CAD configuration converted to a Standard Tessellation Language (STL) format and programmed into the controller). The printing head may include a plurality of jetting nozzles. The jetting nozzles dispense material onto the receiving medium to create the layers representing cross sections of a 3D object.

In addition to the printing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for cooling the dispensed building material for contacting it with a reagent that promotes curing.

Additionally, the printing system may include a leveling device for leveling and/or establishing the height of each layer after deposition and at least partial solidification, prior to the deposition of a subsequent layer.

According to the present embodiments, the additive manufacturing method described herein is for bioprinting a biological object.

Herein throughout, the phrase “modeling material formulation”, which is also referred to herein interchangeably as “modeling formulation” or “modeling material composition” or “modeling composition”, describes a part or all of the uncured building material which is dispensed so as to form the object, as described herein. The modeling formulation is an uncured modeling formulation, which, upon exposure to a curing condition, forms the object or a part thereof.

In the context of bioprinting, an uncured building material comprises at least one modeling formulation that comprises one or more cells (e.g., tumor cells and microenvironment thereof, as defined herein, e.g., astrocytes and microglia) as described herein, and is also referred to herein and in the art as “bio-ink” or “bio-ink formulation”.

In some embodiments, the bioprinting comprises sequential formation of a plurality of layers of the uncured building material in a configured pattern, preferably according to a three-dimensional printing data, as described herein. At least one, and preferably most or all, of the formed layers comprise(s) a cellular component, preferably a plurality of cellular components, as described herein. Optionally, at least one of the formed layers comprises one or more curable materials, preferably biocompatible curable materials which do not interfere with the biological and/or structural features of the cellular components in the bio-ink. According to some embodiments some of the modeling formulations do not comprise cells when printed.

According to an aspect of some embodiments of the present invention the 3D model is designed according to imaging data of the natural tumor. Thus according to some embodiments, the method comprises imaging the tumor to acquire a 3D model of the tumor and optionally a surrounding environment of the tumor, that is, for example, employing a three-dimensional medical imaging technique to thereby acquire a three-dimensional imaging data of the tumor and optionally its surrounding environment (ROI);
ex-vivo dissociating the tumor and optionally its surrounding environment so as to obtain a cell suspension comprising a plurality of cell types; and
subjecting the cell suspension to bioprinting according to the 3D model (or 3D imaging data) so as to obtain a 3D model of the tumor (and optionally its surrounding environment). The latter step and optionally the dissociation step are present regardless of whether the method is based on 3D imaging of the natural tumor.

In some embodiments, the bioprinting comprises receiving 3D printing data and forming the layers in accordance with the 3D printing data, whereby the 3D printing data is generated based on the 3D imaging data. Thus, the 3D model of the tumor features a 3D arrangement (structure, architecture) that has at least 70%, at least 80%, at least 90%, at least 95%, at least 98%, at least 99% or higher, match with the 3D imaging data.

Determining a match to the 3D imaging data can be made by determining the % of voxels in the bioprinted tumor that are identical to voxels of the 3D imaging data and/or comparing other coordinates or parameters of the bioprinted tumor model to corresponding coordinates and/or parameters of the 3D imaging data.

Alternatively, or in addition, the matchability to the 3D imaging data can be determined by the quality of the polymeric scaffold and its ability to mimic the anatomical structure of the tumor. The parameters tested for validation include, for example, swelling capabilities, elasticity, mechanical strength, porosity, etc. Methods of determining such parameters are well-known to those skilled in the art and some exemplary methods are described in the Examples section that follows.

In some embodiments, imaging the tumor is effected using a medical imaging technique as described herein. The imaging can be effected in vivo or ex vivo (upon dissecting the tumor or a portion thereof).

The bioprinting method described herein meets an essential requirement for reproducing the complex, heterogeneous architecture of the tumor upon a comprehensive understanding of the composition and organization of its components. This is achieved by utilizing medical imaging technologies/techniques which can provide the required information on 3D structure and function at the cellular, tissue, organ and organism levels. These technologies include most noninvasive imaging modalities, the common being computed tomography (CT), or μCT, and magnetic resonance imaging (MRI), or μMRI, though other imaging technologies can be used e.g., ultrasound, X-ray. Computer-aided design and computer-aided manufacturing (CAD-CAM) tools and mathematical modeling are also used to collect and digitize the complex tomographic and architectural information for tissues (3). For example, MRI/CT imaging is used to acquire an accurate digital 3D model of the region of interest (ROI) of the patient's tumor and its surrounding microenvironment. MRI provides high spatial resolution in soft tissue, with the advantage of increased contrast resolution, which is useful for imaging soft tissues in close proximity to each other.

In some embodiments, at least a portion of the tumor is removed from a subject and is thereafter dissociated, such that the method comprises, prior to dissociating the tumor, removing a portion of the tumor, and a surrounding environment of the tumor, from a subject. This can be done by means of a surgery, a biopsy, and any other acceptable means. Obtaining the 3D imaging data can be made prior to or subsequent to removing the tumor or a portion thereof.

In some embodiments, dissociating the tumor (or a portion thereof and/or a surrounding environment thereof) is effected by enzymatic dissociation and/or mechanical dissociation.

The obtained cell suspension is then used as a bio-ink or a part thereof as described herein in a selected bioprinting method and a corresponding bioprinting system, for example, as described herein, in combination with one or more acellular curable materials, for example as described herein.

In some embodiments, the bioprinting comprises transferring the obtained 3D imaging data to a 3D printing data readable by a bioprinting system usable in the bioprinting.

In some embodiments, the bioprinting comprises sequentially forming a plurality of layers on a receiving medium in a configured pattern corresponding to the 3D printing data, such that at least one of the layers comprises cells of the cell suspension.

In some embodiments, at least one of the layers comprises a synthetic curable material, or a curable material that forms a synthetic material, as described herein, upon exposure to a curing condition as described herein.

In some embodiments, the curable material is an acellular curable material.

In some embodiments, the curable (e.g., synthetic) material and the 3D printing data are selected or designed so as to provide a chemical, physical and/or mechanical property to the 3D tumor model. In some embodiments, the bioprinting further comprises exposing at least one layer which comprises the curable material to a curing condition (e.g., curing energy), to thereby provide a hardened synthetic (e.g., exogenous and/or acellular, as defined herein) material.

In some embodiments, the hardened synthetic material provides a chemical, physical and/or mechanical property to the 3D tumor model.

In some embodiments, the curable material (e.g., which provides a hardened synthetic material, preferably an exogenous material) and the 3D printing data are selected so as to provide a chemical, physical and/or mechanical property at a pre-determined target location in the 3D tumor model, in accordance with the printing data.

In some embodiments, a method as described herein further comprises characterizing the obtained tumor model, for example, by isolating cells of the tumor model; and in vitro or in vivo culturing the cells.

In some of any of the embodiments described herein, the bioprinting method is configured to effect formation of the layers under conditions that do not significantly affect structural and/or functional properties of the cellular components in the bio-ink.

In some embodiments, a bioprinting system for effecting a bioprinting process/method as described herein is configured so as to allow formation of the layers under conditions that do not significantly affect structural and/or functional properties of the cellular components in the bio-ink.

In some embodiments, the acellular curable materials and/or the curing condition applied to effect curing are selected such that they do not significantly affect structural and/or functional properties of the cellular components in the bio-ink.

### Bioprinting Techniques:

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and case-of-use.

Exemplary suitable bioprinting systems usually contain a temperature-controlled material handling with a dispensing system and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

In some embodiments, the printing provides a printed tumor featuring a plurality of voxel blocks, and at least 70%, at least 80%, at least 90%, or more, as described herein, of these voxel blocks are identical to corresponding voxel blocks of the 3D imaging data used for generating the 3D printing data.

Generally, bioprinting can be effected using any of the known techniques for additive manufacturing. The following lists some exemplary additive manufacturing techniques, although any other technique is contemplated.

### 3D Inkjet Printing:

3D Inkjet printing is the most commonly used type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received (7). Inkjet printers can be used with several types of ink i.e., to use multiple types of cells and ECMs as well as multiple bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic (8, 9) or mechanical (10, 11) dispensing systems. The main advantage of this technique is the ability to deposit very high cell densities. Extrusion bioprinters have been used to construct multiple tissue types, amongst them aortic valves and branched vascular trees as well as for in-vitro pharmacokinetic profiles and tumor modeling (3). The downside of extrusion bioprinting is that fabrication time is relatively slow when printing high-resolution complexed structures.

### Laser-Assisted Printing:

Laser-assisted printing (also known as laser-assisted stereolithography) technique is based on the principle of laser-induced forward transfer, which was developed to transfer metals and is now successfully applied to biological materials. The device consists of a laser beam, a focusing system, a ribbon that has a donor transport support (usually made of glass) that is covered with a laser energy absorbing layer (e.g., gold or titanium), a biological material layer (e.g., cells and/or hydrogel) and a receiving substrate facing the ribbon (3). A laser assisted printer operates by shooting a laser or a binding material at a bed of powder and solidifying it in a highly specific pattern. As the laser or binding agent moves through the powder, layer by layer, it builds a solid structure embedded in powder, which is dusted off when the job is done (3).

Laser associated printing is compatible with a series of viscosities and can print mammalian cells without affecting cell viability or cell function. Cells can be deposit at a density of up to 108 cells/ml with microscale resolution of a single cell per drop (12, 13).

### Electrospinning:

Electrospinning is a fiber production technique, which uses electric force to draw charged threads of polymer solutions, or polymer melts. This cell-laden printing could provide an approach to create small diameter capillary-like blood vessels (14). Another printing technique uses a supporting bath which contains sacrificial hydrogel as a thermoreversible mold to embed the printing of the desired structure from another hydrogel (15). The supporting bath can be made of the Pluronic family of hydrogels or Gelatin.

In some of any of the embodiments described herein, the bioprinting comprises, or consists of, 3D-inkjet printing, as is well-known in the art and is described herein.

Additional bioprinting methodologies include, for example, SWIFT (sacrificial writing into functional tissue), that allows the 3D printing of vascular channels into living matrices composed of organ building blocks (OBBs), as described, for example, in Cartola V. Harvard researchers develop new technique to create human organs. 3D Natives. 2019; Skylar-Scott et al. Sci. Adv. 2019; 5: eaaw2459; and Uijung Yong et al. Prog. Biomed. Eng. 2020; 2:042003; and freeform reversible embedding of suspended hydrogels (known as FRESH), which uses a thermoreversible support bath to enable deposition of hydrogels in complex, 3D biological structures and involves deposition and embedding of the hydrogel(s) being printed within a second hydrogel support bath that maintains the intended structure during the print process and significantly improves print fidelity, such as described, for example, in Hinton et al., Sci. Adv. 1(9), e1500758 (2015); Lec et al., Science 365(6452), 482-487 (2019); Bhattacharjce et al., Sci. Adv. 1(8), e1500655 (2015); and Shiwarski et al., APL Bioeng. 2021 March; 5(1):010904.

Any other bioprinting methodology is contemplated in the context of the present embodiments.

### Modeling Material Formulations (Bio-Ink):

According to some embodiments of the invention, the modeling formulations as used herein are distinct from one another. The first comprises a first synthetic material which serves as the tumor bio-ink, in this case, a combination of fibrinogen, gelatin, thrombin and Transglutaminase (TG) to which the tumor and stroma cells are added. The second comprises Pluronic e.g., F127 and optionally thrombin and serves as the vascular bio-ink.

Thus, at least in the case of the tumor bio-ink, according to some of the present embodiments, it comprises a cell suspension comprising a plurality of cellular components.

In some embodiments, the bio-ink further comprises one or more curable materials e.g., gelatin and fibrinogen.

In some embodiments, the curable material is, or is selected so as to form, a synthetic material as defined herein (e.g., acellular; exogenous; non-biological material as defined herein).

In some embodiments, the bio-ink further comprises a non-cellular (acellular) curable material that forms, upon curing, a synthetic (e.g., exogenous) material as defined herein.

Herein throughout, a “curable material” is a compound (monomeric or oligomeric or polymeric compound) which, when exposed to a curing condition, as described herein, solidifies or hardens to form a cured modeling material as defined herein. Curable materials are typically polymerizable materials, which undergo polymerization and/or cross-linking when exposed to a suitable curing condition or a suitable curing energy (a suitable energy source). Alternatively, curable materials are thermo-responsive materials, which solidify or harden upon exposure to a temperature change (e.g., heating or cooling). Optionally, curable materials are made of small particles (e.g., nanoparticles or nanoclays) which can undergo curing to form a hardened material. Further optionally, curable materials are biological materials which undergo a reaction to form a hardened or solid material upon a biological reaction (e.g., an enzymatically-catalyzed reaction).

In some embodiments, a “curing condition” encompasses a curing energy (e.g., temperature, radiation) and/or a material or reagent that promotes curing (e.g., an enzyme or an analyte).

In some of any of the embodiments described herein, a curable material is a photopolymerizable material, which polymerizes or undergoes cross-linking upon exposure to radiation, as described herein, and in some embodiments the curable material is a UV-curable material, which polymerizes or undergoes cross-linking upon exposure to UV-vis radiation, as described herein.

In some of any of the embodiments described herein, a curable material can be a monomer, an oligomer or a short-chain polymer, each being polymerizable as described herein.

In some of any of the embodiments described herein, when a curable material is exposed to a curing condition (e.g., radiation, reagent), it polymerizes by any one, or combination, of chain elongation or entanglements and cross-linking. The cross-linking can be chemical and/or physical.

In some of any of the embodiments described herein, a curable material is a monomer or a mixture of monomers which can form a polymeric modeling material upon a polymerization reaction, when exposed to a curing condition at which the polymerization reaction occurs. Such curable materials are also referred to herein as monomeric curable materials.

In some of any of the embodiments described herein, a curable material is an oligomer or a mixture of oligomers which can form a polymeric modeling material upon a polymerization reaction, when exposed to a curing condition at which the polymerization reaction occurs. Such curable materials are also referred to herein as oligomeric curable materials.

In some of any of the embodiments described herein, a curable material is or comprises a hydrogel, as defined herein, which can form a hardened modeling material, typically upon further cross-linking and/or co-polymerization, when exposed to a curing condition at which the cross-linking and/or co-polymerization reaction occurs. Such curable materials are also referred to herein as hydrogel curable materials.

In some of any of the embodiments described herein, a curable material is or comprises a hydrogel forming material, as defined herein, which can form a hydrogel as a hardened modeling material, typically upon cross-linking, polymerization and/or co-polymerization, when exposed to a curing condition at which the cross-linking, polymerization and/or co-polymerization reaction occurs. Such curable materials are also referred to herein as hydrogel-forming curable materials.

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of natural and/or synthetic polymeric chains, within the liquid dispersing medium. According to some embodiments of the present invention, a hydrogel may contain polymeric chains of various lengths and chemical compositions, depending on the precursors used for preparing it. The polymeric chains can be made of monomers, oligomers, block-polymeric units, which are inter-connected (crosslinked) by chemical bonds (covalent, hydrogen and ionic/complex/metallic bonds, typically covalent bonds). The network-forming material comprises either small aggregating molecules, particles, or polymers that form extended elongated structures with interconnections (the crosslinks) between the segments. The crosslinks can be in the form of covalent bonds, coordinative, electrostatic, hydrophobic, or dipole-dipole interactions or chain entanglements between the network segments. In the context of the present embodiments, the polymeric chains are preferably hydrophilic in nature.

The hydrogel, according to embodiments of the present invention, can be of biological origin or synthetically prepared.

According to some embodiments of the present invention, the hydrogel is biocompatible, and is such that when a biological moiety is impregnated or accumulated therein, an activity of the biological moiety is maintained, that is, a change in an activity of the biological moiety is no more than 30%, or no more than 20%, or no more than 10%, compared to an activity of the biological moiety in a physiological medium.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly (ethylene glycol), functionalized by cross-linking groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N-(N-propyl)acrylamide), poly(methacyclic acid), poly(2-hydroxyacrylamide), poly (ethylene glycol) acrylate, poly (ethylene glycol) methacrylate, polyvinylalcohol (PVA) and polysaccharides such as dextran, alginate, agarose, and the like, and any co-polymer of the foregoing.

Hydrogel precursors (hydrogel-forming materials) forming such polymeric chains are contemplated, including any combination thereof.

Hydrogels are typically formed of, or are formed in the presence of, di-or tri-or multi-functional monomers, oligomer or polymers, which are collectively referred to as hydrogel precursors or hydrogel-forming agents or hydrogen-forming materials, having two, three or more polymerizable groups. The presence of more than one polymerizable group renders such precursors cross-linkable, and allow the formation of the three-dimensional network.

Exemplary cross-linkable monomers include, without limitation, the family of di- and triacrylates monomers, which have two or three polymerizable functionalities, one of which can be regarded as a cross-linkable functional group. Exemplary diacrylates monomers include, without limitation, methylene diacrylate, and the family of poly (ethylene glycol), dimethacrylate (nEGDMA). Exemplary triacrylates monomers include, without limitation, trimethylolpropane triacrylate, pentaerythritol triacrylate, tris (2-hydroxy ethyl) isocyanurate triacrylate, isocyanuric acid tris(2-acryloyloxyethyl) ester, ethoxylated trimethylolpropane triacrylate, pentaerythrityl triacrylate and glycerol triacrylate, phosphinylidynetris (oxyethylene) triacrylate.

Hydrogels may take a physical form that ranges from soft, brittle and weak to hard, elastic and tough material. Soft hydrogels may be characterized by rheological parameters including clastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

A hydrogel, according to some embodiments of the present invention, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, gelatin acrylate or methacrylate, alginate, alginate acrylate or methacrylate, chitosan, chitosan acrylate or methacrylate, collagen, collagen methacrylate glycol chitosan, glycol chitosan acrylate or methacrylate, hyaluronic acid (HA), HA acrylate or methacrylate, polyethylene-di-crylate (PEGDA) and other non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

In some embodiments, the hydrogel is porous and in some embodiments, at least a portion of the pores in the hydrogel are nanopores, having an average volume at the nanoscale range. In some of any of the embodiments described herein, a curable material, whether monomeric or oligomeric, can be a mono-functional curable material or a multi-functional curable material.

Herein, a mono-functional curable material comprises one functional group that can undergo polymerization when exposed to a curing condition (e.g., radiation, presence of calcium ions).

A multi-functional curable material comprises two or more, e.g., 2, 3, 4 or more, functional groups that can undergo polymerization when exposed to curing energy. Multi-functional curable materials can be, for example, di-functional, tri-functional or tetra-functional curable materials, which comprise 2, 3 or 4 groups that can undergo polymerization, respectively. The two or more functional groups in a multi-functional curable material are typically linked to one another by a linking moiety, as defined herein. When the linking moiety is an oligomeric moiety, the multi-functional group is an oligomeric multi-functional curable material.

In some embodiments, curable materials are printed as a scaffold (optionally a sacrificial scaffold, as in a support material) and a cellular formulation (cell-containing formulation) is printed in and/or on the scaffold. In some embodiments, one or more formulations in the building formulation comprises a mixture of cellular formulation(s) (e.g., a mixture of cells) and (e.g., acellular) curable materials, and the curable (e.g., acellular) materials can be a support material or a model material.

In some embodiments, the tumor model (the object) is made of both cellular and curable (e.g., acellular, synthetic, exogenous) modeling materials, and is formed by forming layers of a building material that comprises a plurality of modeling formulations which comprise cellular components (e.g., tumor cells and additional components from its microenvironment, as defined herein) and curable (acellular; synthetic) materials, optionally in combination with acellular (synthetic) curable support material formulations.

The selection of acellular/synthetic (e.g., curable) materials that will compose the bio-ink, in addition to cell suspensions and/or cellular components, in the design of 3D constructs for tissue engineering applications should be made while considering parameters such as biocompatibility, biodegradability, and cell-substrate interactions.

The bio-inks must flow through the deposition nozzle without clogging, yet should solidify (harden) quickly (e.g., within a time period of no more than a few minutes). Hence, the ink is preferably both shear thinning and viscoelastic, i.e., with a shear elastic modulus (G′) that exceeds the loss modulus (G″).

According to some embodiments of the present invention, the bio-ink (e.g., the one or more modeling material formulation(s)) comprises cellular components, as described herein, and may further comprise curable (e.g., acellular, exogenous/synthetic) components, as described herein.

In some embodiments, the curable material(s) are selected so as to provide the tumor model with chemical, mechanical and/or physical properties that correspond to the respective properties of the tumor, as explained hereinafter.

Curable materials usable in the field of bioprinting are predominantly based on either naturally derived materials (including, for example, Matrigel, Alginate, Pectin, Xanthan gum, Gelatin, Collagen, Chitosan, Fibrin, Cellulose and Hyaluronic acid, often isolated from animal or human tissues) or synthetically-prepared materials (including, for example, polyethyleneglycol; PEG, gelatin methacrylate; GelMA, poly (propylene oxide); PPO, poly (ethylene oxide); PEO), all of which are referred to herein as curable materials that form a synthetic material. Naturally derived materials for 3D bioprinting are advantageous due their similarity to human ECM, and their inherent bioactivity. Synthetically-prepared materials are advantageous in that they can be tailored with specific physical and/or mechanical properties to suit particular applications.

In some embodiments, a curable material, whether it is naturally derived or synthetically-prepared, is a material that forms, upon curing, a synthetic material as described herein (e.g., a material exogenous to the tumor and its environment and/or the subject).

Synthetic materials and/or curable materials forming synthetic materials as described herein can be degradable or non-degradable materials, and may include, for example, hydrogels made of one or more polymers (PEG, polyethyleneglycol-diacrylate, polyglutamic acid, gelatin methacrylate; GelMA, poly (propylene oxide); PPO, poly (ethylene oxide); PEO, PLGA/PLLA), poly(dimethyl siloxane); Nanocellulose; Pluronic F127, or short di-peptides (FF) and Fmoc-peptide-based hydrogel (Fmoc-FF-OH, Fmoc-FRGD-OH, Fmoc-RGDF-OH, Fmoc-2-Nal-OH, Fmoc-FG-OH). Thermoplastic polymers such as Polycaprolactone (PCL), Polylactic acid (PLA) or Poly(D,L-lactide-co-glycolide) along with silicone inks can be used to create customized templates and molds.

The following describes exemplary curable materials usable in the context of the present embodiments:

Gelatin is a low-cost, abundant and biocompatible material, composed of hydrolyzed collagen. The amino acid content of hydrolyzed collagen is the same as collagen.

The term is meant to encompass also analogs of gelatin such as Gelatin methacrylate (GelMA). GelMa is a low-cost, abundant and biocompatible material, composed of denatured collagen that is modified so as to undergo cross-linking when exposed irradiation, preferably in the presence of a photoinitiator. Gelatin is modified with photopolymerizable methacrylate (MA) groups, resulting in a matrix that can be cross-linked through free radical polymerization by short exposure to UV light after printing. By modulating the concentration, degree of methacrylation, and temperature, the shear yield stress and elastic modulus of cured GelMA-containing formulations can be tuned.

Pluronic® materials are class of triblock co-polymers based on Poly-ethylene oxide and Poly-propylene oxide which exhibit reverse thermal gelation. For example, Pluronic F127 is fluid at a low temperature forms a gel at a high temperature, above critical micellar concentration (CMC). Pluronic F127 can be used either as a sacrificial (support) material or be mixed with cellular components. Pluronic F127-diacrylate (DA) is also UV-curable and can be used as an integral part of the final structure.

Fibrin is a glycoprotein in vertebrates that has an important role in the formation of blood clots. Fibrinogen can form a gel when mixed with thrombin, to form fibrin gel. However, fibrin suffers from two main limitations: (i) it has quite poor mechanical properties, and (ii) its gelation process can be too fast from a printing prospective. Therefore, there is a need for a special core-sheath nozzle or post-crosslinking process to avoid gelation prior to extrusion combined with a thickener agent such as pure gelatin which can afterwards be crosslinked to the fibrin gel with Transglutaminase (TG) or some anionic polysaccharide such as Alginate, Xanthan gum or Pectin which can afterwards be crosslinked when the gel is inserted to a cell-media. Combination of PLLA/PLGA sponges with fibrin matrices provides additional mechanical strength (23).

Clay mineral and carbon nanotubes can be included in each of the materials mentioned above to improve the mechanical properties of soft hydrogels and grant electrical properties which can be beneficial to modeling of brain tumors.

According to some embodiments of the present invention, the curable materials in the building material formulation (bio-ink) were selected so as to provide the tumor model with chemical, mechanical and/or physical properties that match the original tumor, as described herein.

For example, for tumors residing in soft tissues such as brain, curable materials that provide synthetic hardened materials exhibiting Young's modulus of about 1-30 kPa are used which is similar to that of GB or metastatic brain tumor.

In exemplary embodiments of the present invention, the bio-ink formulation comprises an enzymatic system, such that the curing is effected by means of one or more enzymatically-catalyzed reactions. The use of such formulations allow controlling the properties of the hardened material by controlling the enzymatically catalyzed reactions, for example, by selecting suitable concentrations of the enzymes.

In exemplary embodiments of the present invention, the tumor bio-ink formulation comprises fibrinogen 0.5-4%, 0.5-3.5%, 0.5-3%, 0.5-2.5%, 0.5-2%, 0.5-1.5%, 1-4%, 1-3%, weight/volume (w/v).

In exemplary embodiments of the present invention, the tumor bio-ink formulation comprises gelatin 1-20% w/v, e.g., 1-18% w/v, 1-16% w/v, 1-14% w/v, 1-12% w/v, 1-10% w/v, 1-8% w/v, 2-20% w/v, 4-20% w/v, 6-20% w/v, 8-20% w/v, 10-20% w/v, 4-8% w/v, e.g., 6% w/v.

According to a specific embodiment, a concentration of the thrombin, if present, in the curable formulation, ranges from 0.1 to 5, or from 0.1 to 4, or from 0.1 to 3, or from 0.1 to 2, or from 0.1 to 1, or is 0.5, U/ml.

According to a specific embodiment, a concentration of the transglutaminase, if present, in the curable formulation, ranges from 0.1 to 50, 0.1 to 10, or from 1 to 10, or from 1 to 5, or is 3, % by volume of the total volume of the formulation.

According to a specific embodiment, the first synthetic material comprises fibrin, gelatin, thrombin and transglutaminase (TG) and CaCl2.

According to a specific embodiment, the tumor bio-ink formulation comprises fibrinogen 0.5-4%, 0.5-4%, 0.5-4%, 0.5-4%, 0.5-4%, 0.5-4%, 0.5-4%, 0.5-4%, 0.5-4%, weight/volume (w/v), e.g., 1% w/v, gelatin 1-20% w/v, e.g., 6%, thrombin 0.05-2 U/ml, e.g., 0.5 U/ml and TG 1-50% w/v. e.g., 3% w/v, the latter used for promoting cross linking of the fibrin by the anionic polymer, i.e., the gelatin.

Optionally, additional acellular agents, curable or non-curable are added to one or more formulations, to further alter the mechanical properties of the hardened material.

In some of any of the embodiments described herein, the bio-ink formulation comprises, in addition to the curable material, a suitable medium for maintaining viability and/or proliferation of the cells in the tumor model.

According to some embodiments, the tumor bio-ink is composed of fibrin, gelatin and thrombin, cross-linked with transglutaminase (TG).

Fibrinogen is combined with the hydrolyzed form of collagen, gelatin, a low-cost, abundant and biocompatible material, and the major component of the ECM in most tissues. Slow crosslinking of the fibrin gel is allowed by transglutaminase (TG), a natural, non-toxic enzyme. TG can catalyze intra-and inter-molecular covalent bonds between glutamine and lysine residues of gelatin.

According to some embodiments, gelatin is prepared by dissolving in phosphate-buffered saline (PBS) without calcium and magnesium under appropriate conditions selected from temperature, e.g., at 70° C. for a few hours e.g., between 2-24 hours e.g., 12 h under stirring. Then, according to some embodiments, the pH is adjusted dropwise to 7.5 such as by using 1 M NaOH. The solution can be filtered through 0.2 μm filter and stored at 4° C. Fibrinogen solution is produced by dissolving lyophilized human blood plasma protein at 37° C. in sterile PBS without calcium and magnesium for a time period sufficient for dissolving, e.g., 45 min. The pH is adjusted dropwise to 7.5 using 0.5 M NaOH and the solution can be stored at −20° C. Transglutaminase (TG) solution (100 mg/ml) is dissolved in PBS without calcium and magnesium, gently mixed for time and temperature (e.g., 20 min at 37° C.) sufficient to obtain a well stirred composition and sterile-filtered. Stock solution of 250 mM CaCl₂ is prepared by dissolving CaCl₂ powder in PBS without calcium and magnesium. To prepare stock solution of thrombin, lyophilized thrombin is reconstituted such as at 2000 U/ml using sterile PBS without calcium and magnesium and stored at −20° C.

According to a specific embodiment, CaCl₂ is present in the tumor bio-ink e.g., 0.01-10 mM. 0.5-10 mM, 1-5 mM, e.g., 2.5 mM.

According to a specific embodiment, a second synthetic material is used to create the vascular bio-ink.

According to some embodiments, the vascular bio-ink is composed of Pluronic F-127 36-38% w/v and optionally thrombin.

It will be appreciated that once fabricated, the 3D model is essentially devoid of the synthetic material (i.e., second synthetic material) from which the vasculature is produced because it is liquified/melted and depleted during the production process.

As used herein “essentially” refers to less than 21% w/v of Pluronic in the 3D model (e.g., less than 15%, 10%, 5%, 1%, 0.1%, 0.01% w/v).

### Microengineered Blood Vessels:

A 3D tumor model as described herein is populated with living cells.

In some embodiments, the method further comprises perfusing the 3D-bioprinted model of the tumor, for example, by creating blood vessels during the bioprinting process, as described in Example 3, herein below.

In some embodiments, the tumor model further comprises small diameter blood vessels.

According to a specific embodiment, the vasculature is coated by the tumor/stroma bio-ink and/or is not in direct contact with the receiving medium (glass).

Achieving vascularization of the desired 3D tumor model, for example, in order to test different drugs on it, is considered a major challenge in bioprinting. Several 3D printers are capable building tiny, hierarchical networks of blood vessels to supply blood.

One approach for achieving vascularization comprises using a customized, high-resolution 3D printer that can form microchannels in biocompatible gels. These hydrogel materials can be printed at the micron-length scale (the smallest microvascular channels that are printed are around 10 microns in diameter). Using this approach, a capillary network of fluorescently labeled sacrificial ink is printed into gel-like matrix which can be melted later. Further, blood vessels can be printed using sacrificial template of carbohydrate glass and seeded with endothelial cells (ECs), such that the ECs line the interiors of the channels and may penetrate the surrounding cell-gel mixture).

In some embodiments, the 3D tumor model described herein comprises a functional perfusable vascular system with active flow, which mimics high pressure pulsatile blood flow, hemodynamics, shear stress, etc. Vascularization is important as it maintains cell viability and encourages tissue organization and differentiation.

In some embodiments of the present invention, a 3D tumor model featuring interconnected channels is manufactured as described herein, and a Pluronic solution, optionally containing, e.g., cells, factors and/or any other biological materials present in the microenvironment of the tumor, is creating the channels, forming a network of microchannels that mimics a vascularized tumor.

An exemplary methodology, which utilizes Pluronic for forming a vascular network in the tumor model is demonstrated in the Examples section that follows.

Thus, a vascular bio-ink is composed of 21-40% w/v Pluronic F-127, 30-40% w/v Pluronic F-127, 35-40% w/v Pluronic F-127 e.g., 38% w/v and optionally thrombin 0-1000 U/ml, 5-1000 U/ml, 10-1000 U/ml, 20-1000 U/ml, 30-1000 U/ml, 40-1000 U/ml, 50-1000 U/ml, 60-1000 U/ml, 70-1000 U/ml, 80-1000 U/ml, 90-1000 U/ml, 100-1000 U/ml, 50-1000 U/ml, 50-500 U/ml, 50-400 U/ml, 50-300 U/ml, 50-200 U/ml, e.g., 100 U/ml.

The 3D printed model may b arranged by printing tumor and stroma cells and vasculature in a layered manner.

### Lymph Node

According to another aspect of the present invention there is provided a cellularized 3D structure, generated by 3D printing, comprising:
at least one inner compartment comprising B cells;
an outer compartment comprising a first cell type of a lymph node; and
an intermediate compartment, between the inner compartment and the outer compartment comprising a second cell type of the lymph node,
at least one inlet port and at least one exit port.

According to a specific embodiment, the cells of the 3D lymph node can be of primary cells, non-immortalized cells, freshly isolated from a patient without any culturing or cloning, cell lines or a combination of same.

Examples of lymph node tissue contemplated by the present inventors include:
Axillary lymph nodes are the lymph nodes located in the armpit (axilla);
Supraclavicular lymph nodes are located just above the collarbone (clavicle);
Inguinal lymph nodes are located in the groin; and
Mesenteric lymph nodes lie deep within the abdomen in the membranes that surround the intestine.

According to another embodiment, the 3D lymph node is generated by 3D printing.

According to a specific embodiment, the cells of the 3D lymph node are mammalian cells e.g., mouse.

According to a specific embodiment, the cells of the 3D lymph node are human cells. According to some embodiment, the cells of the 3D lymph node are from a single host (e.g., at least the lymphocytes).

According to a specific embodiment, the cells of the 3D lymph node are from different hosts.

According to a specific embodiment, the cells of the 3D lymph node are from different hosts (e.g., different human beings) or organism origin e.g., human and mouse.

According to a specific embodiment, each of the plurality of cell types of the 3D lymph node is from different organisms (e.g., different patients).

According to a specific embodiment, some of the plurality of cell types are from different organisms (e.g., different patients, e.g., the lymphocytes are from one organism and the vascularization cells are from other(s)).

According to a specific embodiment, the 3D lymph node comprises at least two different cell types, wherein one of the cell types is a B cell and the other cell type is selected from T cells, dendritic cells and macrophages. For example the 3D lymph node may comprise B cells T cells and dendritic cells, or B cells dendritic cells and macrophages. Optionally, the 3D lymph node may further comprise tumor cells. The tumor cells may be derived from the same patient as the lymphocytes are derived.

According to a specific embodiment, each of the plurality of cell types is from the same organism.

According to a specific embodiment, all the cells are autologous to the subject on which personalized screening for drugs will take place using the 3D model (e.g., full HLA matchability as described above).

Methods of 3D printing lymph nodes are described herein above with respect to printing of tumor models and are relevant to generation of lymph nodes as well. The 3D printed lymph node typically comprises at least one synthetic material, as detailed herein above with respect to 3D printed tumor models.

According to a particular embodiment, the bioink used to print the 3D lymph node comprises at least one of fibrin, collagen, laminin, fibronectin, locust beam gum and gelatin.

In one embodiment, the bioink comprises collagen, fibronectin, laminin, transglutaminase (TG) and gelatin.

In another embodiment, the bioink comprises collagen, fibronectin, laminin and locust bean gum.

In still further embodiments, the bioink comprises collagen (e.g. 1-5 mg/ml), fibronectin (e.g. between 50-200 ug/ml) gelatin (e.g. between 0.5-5%).

According to another embodiment, the bioink used to print the 3D lymph node comprises

In one embodiment, the lymph node is bioprinted in a mold.

The term “mold” refers to an object which comprises walls which define the shape and dimensions of a liquid which is poured therein following polymerization.

The mold comprises sturdy and/or rigid sidewalls made from materials such as metal, plastic, or silicone. These walls define the shape and dimensions of the object being produced.

In one embodiment, the mold is shaped to mimic the natural shape of a lymph node (e.g. bean shaped or kidney shaped), whereby one surface of the mold is concave and the opposing surface is convex.

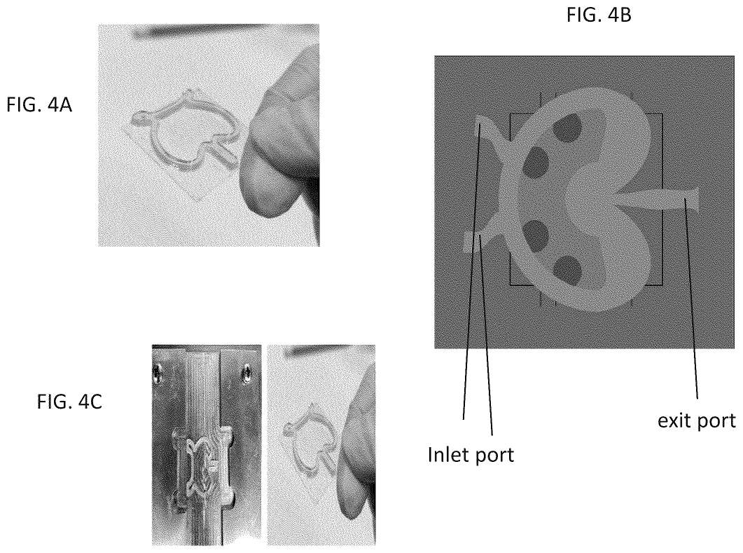

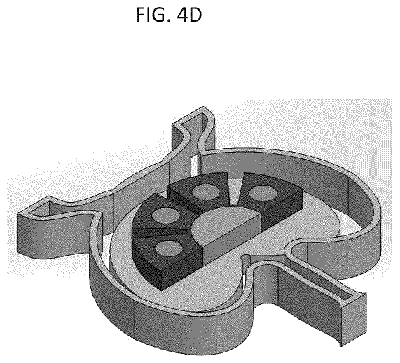

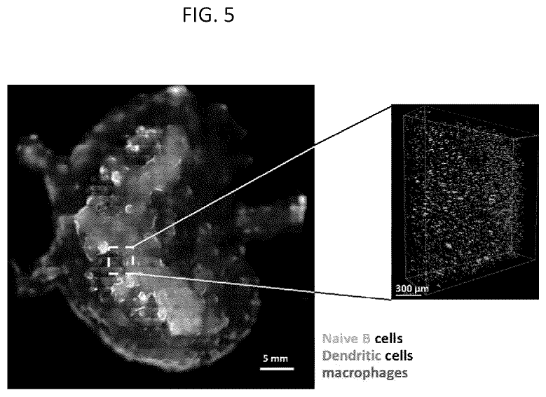

Both the concave surface of the mold and the convex surface of the mold may further comprise at least one extension which serves as an entry and/or exit port to the lymph node—see FIG. 4B for example. The entry port mirrors the efferent lymphatic vessel of the lymph node and the exit port may mirror the afferent lymphatic vessel of the lymph node or vice versa. In a particular embodiment, the mold is designed to include at least two extensions on the convex surface (e.g. serving as two inlet ports) and further to include at least one extension of the concave surface (e.g. serving as an exit port). The extensions typically include an opening to allow fluid to perfusion the printed tissue.

A typical surface area of the mold is between 0.5 cm to 5 cm. The entry and exit ports length are between 0.1 to 0.5 cm. The depth of the mold is typically between 0.1 to 2 cm.

Typically the mold is fabricated from an elastomer. Preferred elastomers of the instant invention are biocompatible, gas permeable, optically clear elastomers including silicone rubbers, e.g. PDMS. Additional examples of elastomers are provided herein above.

Printing of the cells inside the mold may be according to a particular pattern. For example, the present inventors contemplate at least one, at least two, at least three, at least four inner areas (compartments) of B cell lymphocytes. The inner areas may be of any shape, e.g. circular. T cells and/or dendritic cells are deposited such that they substantially surround the areas of B cells. The inner compartments have at least 5 times or even at least 10 times the amount of B cell lymphocytes as T cell lymphocytes, macrophages or dendritic cells. The T cells and/or dendritic cells may be deposited in a particular shape (e.g. C shape), as illustrated in FIG. 4B. The C-shaped compartment typically comprises at least 5 times or even at least 10 times the amount of T cell lymphocytes, macrophages or dendritic cells as compared to B cell lymphocytes. Additional cells are then added to fill the mold-e.g. macrophages and/or dendritic cells. The additional cells which are used to fabricate the surrounding arca typically comprise at least 5 times or even at least 10 times the amount of T cell lymphocytes, macrophages or dendritic cells as compared to B cell lymphocytes. The cells used to farbricate the lymph node may be derived from a single subject or from multiple subjects. The subject may be a healthy subject or have a disease which affects the lymph node (e.g. cancer). In one embodiment, the cells of the lymph node comprise cancer cells which have metastasized to the lymph node.

Following deposition, and optional curing, the tissue is cultured in a suitable culture medium such as a mixture of DMEM cell media together with Endothelial cell media, pericyte cell media, astrocytes cell media, microglia cell media, T cell media, B cell media, in a 1:1:1:1: . . . ratio or only T cell media, or only DMEM cell media, or any other 1:1 ration depending on the cell type composition.

In one embodiment, the 3D printed lymph node is cultured in a perfusion system (e.g. such as described herein above). Organization of the 3D printed lymph node in a perfusion system may be such that fluid flows from tumor cells to the lymph node (or vice versa) as described herein above. The 3D printed lymph node may be useful for selection of drug candidates or for basic research, as further described herein below.

### Perfusing the Perfusion System or the 3D Printed Lymph Node

The culture wells of the perfusion system or the 3D printed lymph node may be perfused with cell culture medium. The cell media is selected according to the cell type which is cultured, examples of which are known to those of skill in the art.

The terms “medium”, “cell culture medium”, “culture medium”, and “growth medium” as used herein refer to a solution containing nutrients which nourish growing eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant polypeptides or proteins, for example, but not limited to, hormones, cytokines, interleukins and other signaling molecules.

In one embodiment, the cell culture medium comprises blood or fractions thereof e.g., PBMC, serum, sub-populations thereof e.g., myeloid cells e.g., macrophages, NK cells, T-cells, which are naturally occurring or engineered such as to express a chimeric T cell receptor (CAR). In one embodiment, the cell culture medium is derived from the same subject from where the cells originate.

According to a particular embodiment, the perfusion system comprises:
a sequence of at least two cell-culture wells comprising:
   a first cell-culture well which comprises a first population of cells derived from a test subject;
   a second cell-culture well which comprises a second population of cells derived from the test subject;
   a fluid pathway interconnecting the first cell-culture well and the second cell-culture well so as to allow for fluid to flow through the sequence in a sequential manner; and
   fluid of the fluid pathway, wherein the fluid is derived from the test subject.

In this embodiment, the cells in the first cell culture well and the second cell culture well are derived from a single test subject (e.g. a diseased subject) and the fluid used to perfuse the cells is also derived from that subject. In one embodiment, the cells in the first cell culture well and the second cell culture well are tumor cells of the subject. The fluid used to perfuse the cells may comprise blood or a fraction thereof derived from the subject (e.g. PBMCs).

The present inventors have shown that using the above described perfusion system enabled the selection of a particular cancer treatment in less than 2 weeks, as described in Example 4, herein below.

### Applications:

The cell perfusion system as described herein is usable in various applications, including research (e.g., for drug design, drug screening, simulating surgery) and for the purpose of evaluating an operative therapeutic agent (e.g. anti-cancer agent) suitable for the specific diseases cells (e.g. tumor cells) which are cultured within.

According to an aspect of some embodiments of the present invention there is provided a method of determining an effect (e.g. a therapeutic effect, a prophylactic effect or a toxic effect) of an agent comprising:
(a) flowing the agent through the cell perfusion system described herein; and
(b) measuring:
   (i) at least one parameter of the fluid; and/or
   (ii) at least one parameter of the cells of the first and/or second population;
   wherein a change in the at least one parameter of the fluid and/or of the cells as compared to the at least one parameter in the absence of the agent, is indicative of an effect of the agent.

Parameters of the fluid which may be measured include, but are not limited to oxygen concentration, glucose concentration, fatty acids composition, electrical resistance, pH, temperature, metabolite concentration and flow rate.

Measuring can be achieved using detectors that are incorporated into the cell perfusion separate or that are separate from the system but aligned with the region of the system to be detected.

A number of different detection strategies can be utilized with the cell perfusion system, examples of which are provided herein. The detectors can be designed to detect a number of different signal types including, but not limited to, signals from fluorophores, chromophores, polypeptides that emit chemiluminescence, electrochemically active polypeptides, enzymes, cofactors, enzymes and enzyme substrates.

Illustrative detection methodologies suitable for use with the present cell perfusion systems include, but are not limited to, light scattering, multichannel fluorescence detection, UV and visible wavelength absorption, luminescence, differential reflectivity, and confocal laser scanning. Additional detection methods that can be used in certain application include scintillation proximity assay techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET). Additional detection options include electrical resistance, resistivity, impedance, and voltage sensing.

The detection section can be in communication with one or more microscopes, diodes, light stimulating devices (e.g., lasers), photomultiplier tubes, processors and combinations of the foregoing, which cooperate to detect a signal associated with a particular event and/or agent. Often the signal being detected is an optical signal that is detected in the detection section by an optical detector. The optical detector can include one or more photodiodes (e.g., avalanche photodiodes), a fiber-optic light guide leading, for example, to a photomultiplier tube, a microscope, and/or a video camera (e.g., a CCD camera).

Detectors can be microfabricated within the cell perfusion system, or can be a separate element. If the detector exists as a separate element and the microfluidic device includes a plurality of detection sections, detection can occur within a single detection section at any given moment. Alternatively, scanning systems can be used. For instance, certain automated systems scan the light source relative to the microfluidic device; other systems scan the emitted light over a detector, or include a multichannel detector. As a specific illustrative example, the microfluidic device can be attached to a translatable stage and scanned under a microscope objective. A signal acquired is then routed to a processor for signal interpretation and processing. Arrays of photomultiplier tubes can also be utilized. Additionally, optical systems that have the capability of collecting signals from all the different detection sections simultaneously while determining the signal from each section can be utilized.

The detector can include a light source for stimulating a reporter that generates a detectable signal. The type of light source utilized depends in part on the nature of the reporter being activated. Suitable light sources include, but are not limited to, lasers, laser diodes and high intensity lamps. If a laser is utilized, the laser can be utilized to scan across a set of detection sections or a single detection section. Laser diodes can be microfabricated into the microfluidic device itself. Alternatively, laser diodes can be fabricated into another device that is placed adjacent to the microfluidic device being utilized to conduct a thermal cycling reaction such that the laser light from the diode is directed into the detection section.

Detection can involve a number of non-optical approaches as well. For example, the detector can also include, for example, a temperature sensor, a conductivity sensor, a potentiometric sensor (e.g., pH electrode) and/or an amperometric sensor (e.g., to monitor oxidation and reduction reactions). A number of commercially-available external detectors can be utilized. Many of these are fluorescent detectors because of the case in preparing fluorescently labeled reagents. Specific examples of detectors that are available include, but are not limited to, Applied Precision ArrayWoRx (Applied Precision, Issaquah, Wash.)).

According to another aspect of some embodiments of the present invention there is provided a method of determining an effect of an agent on the 3D cellularized lymph node structure described herein comprising subjecting the 3D lymph node structure as described herein (with or without perfusion as described herein) of a lymph node as described herein to a therapeutic treatment regimen; and determining a presence of a therapeutic effect (e.g., inhibition of tumor growth, killing of cancer cells, inducing apoptosis of cancer cells, anti-angiogenic effect) of the treatment regimen on the lymph node cells.

Thus, according to a specific embodiment, subjecting refers to contacting for a predetermined time period.

According to another embodiment, subjecting refers to perfusing such as using the system as described herein.

Perfusion can be of biological samples such as blood or fractions thereof e.g., PBMC, serum, sub-populations thereof e.g., myeloid cells e.g., macrophages, NK cells, T-cells, which are naturally occurring or engineered such as to express a chimeric T cell receptor (CAR).

Perfusion can be of small molecules or biological molecules such as polypeptides, peptides, antibodies, nucleic acid agents (e.g., RNA silencing agent, DNA/RNA editing agents), carbohydrates, lipids or combinations of same.

According to a specific embodiment, the perfused substance is an FDA-approved drug.

According to a specific embodiment, the perfused substance is a research/test molecule.

According to some embodiments, the perfused substance is chemically defined (with above 95% certainty regarding the identity of the chemical content).

According to some embodiments, the perfused substance comprises a potential prophylactic effect (e.g. an anti-cancer vaccine, such as a dendritic cell-targeted vaccine including nanoparticles which comprise at least one of the following components mRNA, RNAi, peptides, TLRs, adjuvants, etc.)

According to some embodiments, the perfused substance is chemically undefined (e.g., blood or fractions thereof or conditioned medium).

Various assays can be used to determine the effect of the anti-cancer agent/regimen on cells. Some non-limiting examples are described herein below.

According to a specific embodiment, the “assay” is a procedure for testing or measuring the presence or activity of a substance (e.g., a chemical, molecule, biochemical, drug, physical condition e.g., radiation, etc.) in cells, including for example cells of the 3D lymph model.

In further embodiments, assays include qualitative assays and quantitative assays. In still further embodiments, a quantitative assay measures the amount of a substance in a sample.

In various embodiments, the assay is selected from the group consisting of an image-based assays, measurement of secreted proteins, expression of markers, and production of proteins.

In various further embodiments, the perfusion system and 3D models as describe herein are for use in assays to detect or measure one or more of: molecular binding (including radioligand binding), molecular uptake, activity (e.g., enzymatic activity and receptor activity, etc.), gene expression, protein expression, receptor agonism, receptor antagonism, cell signaling, apoptosis, chemosensitivity, transfection, cell migration, chemotaxis, cell viability and apoptosis, metabolic activity, cell proliferation, safety, efficacy, metabolism, toxicity, and abuse liability.

In various further embodiments, the 3D models and perfusion systems as describe herein are for use in immunoassays. In further embodiments, immunoassays are competitive immunoassays or noncompetitive immunoassays. In a competitive immunoassay, for example, the antigen in a sample competes with labeled antigen to bind with antibodies and the amount of labeled antigen bound to the antibody site is then measured. In a noncompetitive immunoassay (also referred to as a “sandwich assay”), for example, antigen in a sample is bound to an antibody site; subsequently, labeled antibody is bound to the antigen and the amount of labeled antibody on the site is then measured.

According to a specific embodiment, an immunoassay assays the effect of immune cells (e.g., autologous or non-autologous e.g., allogeneic) on the tested cells (e.g. those in the perfusion system and those in the lymph node model). Such cells can be obtained from the blood e.g., PBMC and tested in the above described system.

Immune cells can include, but are not limited to, the innate immune cells, adaptive immune cells or components thereof.

The immune cells can be provided in a biological sample (e.g., serum) or alternatively in a culture medium.

It will be appreciated that the effect of various factors in a medium can be tested also in the absence of immune cells.

In various further embodiments, the perfusion systems and 3D lymph models as described herein are for use in drug screening or drug discovery. In further embodiments the perfusion systems and 3D lymph model are used as part of a kit for drug screening or drug discovery. In some embodiments, each 3D lymph model exists within a well of a biocompatible multi-well container, wherein the container is compatible with one or more automated drug screening procedures and/or devices. In further embodiments, automated drug screening procedures and/or devices include any suitable procedure or device that is computer or robot-assisted.

In various further embodiments, the perfusion systems and 3D lymph models as described herein are for use in research or develop drugs potentially useful in any therapeutic area including anti-cancer efficacy, pharmacology, toxicology, and immunology.

In the case where the perfusion system comprises a healthy organ model (as described herein above) or where the 3D lymph node model is connected to a healthy organ model (as described herein above), the system may be used to analyze the side effect of agents which are perfused through the system on healthy organs.

In a particular embodiment, the perfusion systems and 3D lymph models as describe herein is for use to identify therapies potentially useful in the disease or condition of a particular individual. In further embodiments, the methods include applying a candidate therapeutic agent or condition to the perfusion systems and 3D lymph model; measuring viability of the cells; and selecting a therapeutic agent for the individual based on the measured viability of the cells. In still further embodiments, the candidate therapeutic agent is a one or more chemotherapeutic compounds, one or more radiopharmaceutical compounds, radiation therapy, immune modulator (e.g., checkpoint modulator) or a combination thereof. Accordingly, disclosed herein are methods of personalizing medicine to a subject in need thereof.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

As used herein the term “about” refers to +10% or +5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Exemplary Materials and Methods That Can Be Used for Generating a Cell Perfusion Device:

Commercial 96 glass bottom well plates (Cellvis, California, USA) were drilled with a driller (e.g. 1.5 mm driller) through an entire column using a CNC machine. A drill jig was used to ensure accurate positioning in Z axis while drilling as close as possible to the bottom of the plate (+0.5 mm) to allow maximal focal penetration imaging and optimal noninvasive monitoring of fluorescent signals. After drilling 12 channels through the plate, condensed air was applied to dispose plastic residues and dust and needles (e.g. 12 Hamilton 25 G, 4-inch length, blunt needles (Hamilton, Romania)) were placed in each channel framed by a polymer (e.g. polydimethylsiloxane (PDMS) (Sylgard™ 184, Dow®, Michigan, USA) on the bottom side of the well plate to allow tight sealing between the 1.5 mm holes and the 25 G needles. A few cycles of PDMS casting were performed to ensure tight sealing.

After the fabrication of the well plate with the channels, the plates may be UV (254 nm) irradiated to allow further sterile work with the plates.

### Exemplary Method 1—Channel Lined With Endothelial Cells Following Generation of 3D Cellular Model

The wells may be cast with a casting formulation (e.g. based on fibrinogen and/or gelatin) with cells (e.g. cancer cells) on top of the needles, covering them completely to create a straight perfusion channel in each well as was previously described [Neufeld L., et al. Science advances 7.34 (2021): eabi9119]. The cells should be incubated under conditions which allow crosslinking (e.g. incubation at 37° C. is required for a minimum of 1 hour) so as to reach the physiological stiffness of the desired tissue of interest.

The needles can then be removed and void channels were formed in each well. The channel was then covered with cells (e.g. endothelial cells and pericytes) of the relevant tissue of interest by injecting a concentrated cell mixture into the vessel. The cells can be incubated for a relevant amount of time (e.g. 2 hours). Steps of this method are illustrated in FIGS. 1A-C. Cell seeding steps can be repeated a number of times (e.g. 2, 3, 4), and each time sample incubation positioning may be rotated (e.g. by 90 degrees) to maximize cell attachment area. The model may then be incubated in rotation overnight at 37° C. to allow cell attachment to the lumens' walls with a full coverage of the lumen.

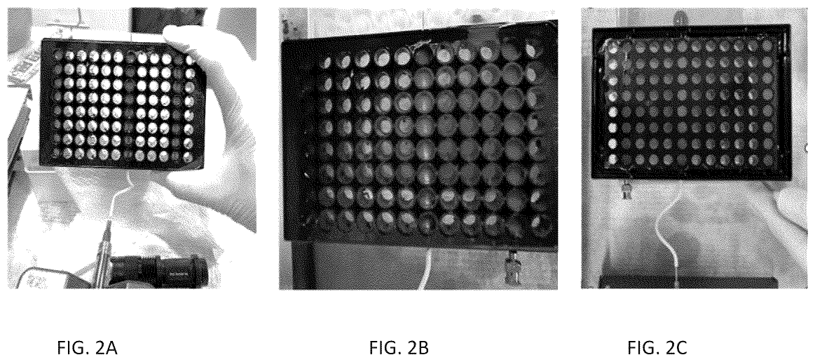

The channels were perfused with food dye to allow its imaging, as depicted in FIGS. 2A-C. The use of the food dye shows the potential to perfuse the sample through the vessels that are used as channels to transport nutrients and waste into and out of the system, respectively. These perfusable blood vessels offer the possibility to mimic the blood brain barrier allowing the evaluation of the integrity and functionality of the endothelial barrier in the presence or absence of cancer cells.

### Exemplary Method 2—Channel Lined With Endothelial Cells Concomitantly With Generation of 3D Cellular Model

A volume of the casting formulation was added just until the height of the hole between the wells (e.g. 0.5 mm). On top of the first casted/printed formulation (e.g. fibrinogen and/or gelatin), 3D-printed vascular channels can be printed using a sacrificial bioink such as Pluronic F127 40% w/v, or 38% w/v Pluronic F127 and 100 U/ml Thrombin or many others potential sacrificial bioinks. On top of the 3D-printed vasculature, a casting formulation was cast to cover the 3D-printed structure. Conditions were selected to allow for sufficient crosslinking, reaching the physiological stiffness of the desired tissue of interest. Then the Pluronic F127 may be washed as previously described by Neufeld et. al., Science advances 7.34 (2021):eabi9119.

### Example 2

### Exemplary Materials And Methods That Can Be Used For 3D-Bioprinting A Lymph Node:

A silicone frame was printed on a glass side that will surround the 3D-bioprinted tumor derived lymph node (TDLN), as illustrated in FIG. 4A. Cells were 3D printed using a bioink based on fibrin-gelatin within the frame according to the design illustrated in FIG. 4B. Each different color corresponds to a different cell type. In one embodiment, blue corresponds to B cells; green corresponds to dendritic cells; and yellow corresponds to macrophages. In another embodiment, blue corresponds to B cells, green corresponds to T cells; and yellow corresponds to dendritic cells. The frame was sealed using a freshly prepared liquid PDMS which was spread on the silicone frame. A second glass slide was placed on top between two magnets (FIG. 4C). Following crosslinking with thrombin and transglutaminase, the 3D-bioprinted construct was perfused with cell media.

An exemplary 3-D bioprinted lymph node is shown in FIG. 5.

### Example 3

### Exemplary Materials and Methods That Can Be Used for 3D-Bioprinting a Vascularized Tumor Model:

### Preparation of the Bioink

Pluronic F127 is a biologically inert triblock co-polymer based on poly-ethylene oxide (PEO) and poly-propylene oxide (PPO), which exhibits reversible thermal gelation and a lower critical solution temperature (LCST) behavior. At lower temperatures, Pluronic F127 solution is liquid, and upon heating to room temperature it transforms into a gel. 40% w/v Pluronic F127 was dissolved in double distilled water (DDW) using an overhead mechanical stirrer at 4° C. The solution was stored at 4° C. Prior to use, 2000 U/ml The solution was added to create the fugitive vascular bioink of final 38% w/v Pluronic F127 and 100 U/ml Th. The bioink was loaded into a 30ml syringe at 4° C. and centrifuged to remove air bubbles.

### 3D Multi-Material Bioprinting

The syringe was placed in its cartridge which was set to 29° C. 3D structures were printed using a 3D-BioPlotter (EnvisionTEC®, Germany) equipped with five independent ink cartridges. The design of patterns was created using EnvisionTEC software. Inks were loaded in separate syringes, and tapered nozzles of varying size were attached via a luer-lock (Nordson). Before each printing, the nozzles are calibrated to determine their respective X, Y and Z offsets. The inks were printed on a plastic film supplied by the manufacturer. Cell-laden GelMA was printed for a time period up to 2 h to limit cell death due to lack of oxygen.

The following conditions were set to print 15% w/v GelMA: extruder temperature (29° C.), platform temperature (15° C.), print head linear speed (22 mm/s), pressure (1.6 bar), needle offset (0.25 mm), and syringe metal tip diameter (0.25 mm). The spacing between fibers was adjusted to achieve distinct pore sizes of 1.75 mm or 0.75 mm. Each printed layer was crosslinked with varied UV light procedures (single or continuous projection) to produce a solid hydrogel with minimal UV exposure (single UV projection for 15 sec with 30 mm height from the last layer). The conditions to print with the fugitive 40% w/v Pluronic F127 are: extruder temperature (29° C.), platform temperature (room temp.), print head linear speed (20 mm/s), pressure (3.5 bar), needle offset (0.3 mm), and syringe metal tip diameter (0.25 mm). The spacing between fibers was adjusted to achieve distinct pore sizes of 1.75 mm or 0.75 mm.

Printing formulation of fibrin 3D-bioink (composed of 1% w/v fibrinogen and 6% w/v gelatin) was mixed with PD-GB4 cells labeled with Azurite (1×10⁶ cells/ml), hAstro labeled with GFP (1×10⁶ cells/ml) and hMG cells (1×10⁵ cells/ml). The mixture forming the GB-bioink was loaded in a syringe (Nordson, California, USA) and tapered with a needle tip (Nordson) of varying size attached via a luer-lock. The GB-bioink was cooled to 4° C. for 15 min and then loaded into the 3D printer's cartridge. Temperature was set to 24° C. and held for 15 min prior to 3D-bioprinting initiation. 3D structures were printed using a 3D-Bioplotter® (Manufacturer series, EnvisionTEC®, Gladbeck, Germany) equipped with five independent ink cartridges. Before each 3D-bioprinting, the nozzles were calibrated to determine their respective X, Y and Z offsets. Following calibration, 6 to 8 layers were printed on a thin coverslip, framed by a polydimethylsiloxane (PDMS) (Sylgard™ 184, Dow®, Michigan, USA) gasket, creating the bottom platform onto which vasculature structure could be printed. Cell-laden bioinks were printed for a time period up to 2 h to prevent cell death. The printed layers were left to dry for up to 1 h. Casting formulation of fibrin 3D-bioink (composed of 1% w/v fibrinogen and 6% w/v or 12% w/v gelatin) was mixed with Azurite-labeled PD-GB4 cells (1×10⁶ cells/ml), GFP-labeled hAstro (1×10⁶ cells/ml) and hMG (1×10⁵ cells/ml). The mixture forming the GB-bioink was casted on a thin coverslip, framed by a PDMS gasket, creating the bottom platform onto which vasculature structure could be printed. Cell-laden fibrin 3D-bioink constructs were left to fully crosslink for up to 2 h. Then, the vascular bioink composed of Pluronic F127 38% w/v and Th 100 U/ml was loaded onto a printer cartridge (Nordson) at 4° C., warmed to 29° C., and printed using a 0.25 mm needle tip attached via a luer-lock. 3D structures were printed with the vascular bioink according to a bioengineered design of vasculature, created with Rhino 6® 3D modeling software (Rhinoceros®). Immediately after the vascular bioink 3D-bioprinting was completed, connectors (Darwin microfluidics, Paris, France) were inserted to the inlet and outlet positions of the PDMS gasket to allow future flow by peristaltic pump, and a fresh formulation of the fibrin 3D-bioink with PD-GB4 and microenvironment cells was casted, covering the printed vasculature and filling the PDMS frame completely. The sample was then covered with additional coverslip glass and sealed in a metal frame (creating the perfusable chip), self-designed for optimal noninvasive monitoring of fluorescent signals and incubated at 37° C. for a minimum of 3 h until complete crosslinking is achieved, reaching the physiological stiffness of the brain. To liquefy the fugitive Pluronic F127 bioink, the sample was cooled to 4° C. Cold PBS was then injected into the mold's inlet by applying positive pressure, while applying negative pressure through the outlet leaving the model with the desired 3D-bioprinted lumens. Following Pluronic F127 wash, fibronectin (100 μg/ml) was injected into the lumens' inlet and incubated in rotation for 3 h at 37° C. to prime the vasculature wall and create an adherable interface. Next, a mixture of mCherry-labeled HUVEC (8×10⁶ cells/ml) and iRFP-labeled human microvascular brain pericytes (2×10⁶ cells/ml) (named vascular bioink) at 4:1 ratio was injected into the vessel and incubated for 2 h. Cell seeding steps were repeated 4 times, and each time sample incubation positioning was rotated by 90 degrees to maximize cell attachment area. The model was then incubated in rotation overnight at 37° C. to allow cell attachment to the lumens' walls with a full coverage of the lumen. The next day the sample was connected to a peristaltic pump (EBERS, Zaragoza, Spain), incubated at 37° C. and perfused with medium mixture of all the cells in the samples at a 1:1:1:1:1 ratio (DMEM, astrocyte medium, microglia medium, pericyte medium and EGM-2) for 5 days. After confluent cover was validated by confocal imaging, 1 and 0.1 mg/ml 70 kDa dextran-FITC was perfused through the vasculature using a syringe pump (Braintree scientific, Braintree, Massachusetts, USA) at a flow rate of 25 μl/min. Dextran-FITC flow-through was imaged by EVOS FL Auto cell imaging system at 20 sec intervals. 3D-printed GB models used for dextran-FITC perfusion were created with unlabeled cancer and stromal cells (hAstro and hMG).

### Example 4

### An Exemplary Use of a Perfusion System According to Embodiments of the Invention:

Blood and tissue samples from a mucosal melanoma, a patient having the following characteristics:
Female, 58, malignant mucosal melanoma.
Tissue site: resected from the groin lymph node

Clinical Neoadjuvant therapy: 4 cycles of Nivolumab resulted in progressive disease (PD), without any positive clinical outcome. This was followed by additional cycles of nivolumab post-surgery as adjuvant therapy resulting in further PD.

96-well 3D tumor organoids were generated from the tumor sample (i.e. tumor cells and microenvironment cells, blood vessels and PBMCs were created from the samples. The tumor organoids were cultured in a plate similar to that shown in FIG. 1A. Each treatment was tested on multiple 3D organoids, each treatment corresponding to a single sequence of connected cell wells.

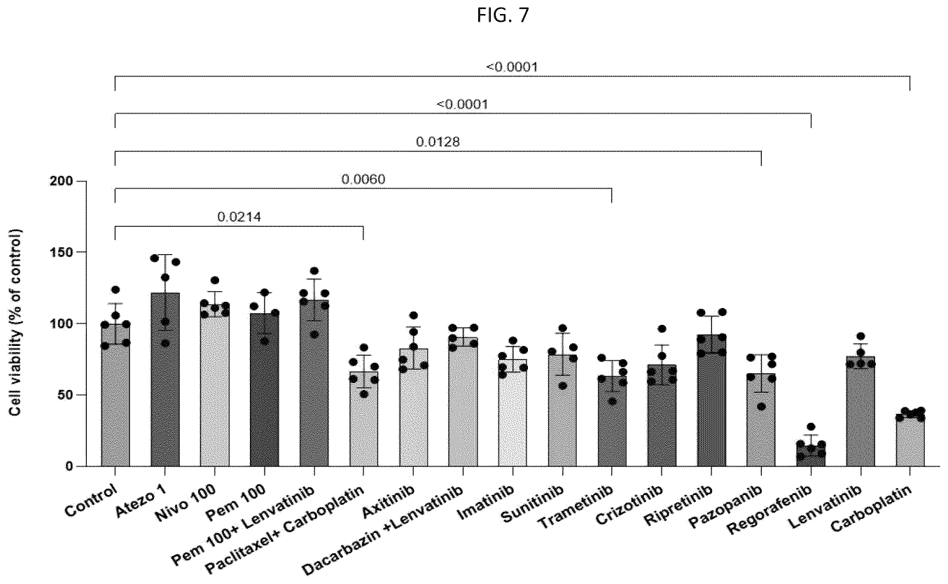

Multiple treatments and combinations were tested on the patient-derived mucosal melanoma cells and the patient-derived PBMCs, as summarized in FIG. 7.

The optimal treatment, according to the model, was found to be Regorafenib, with a high efficacy of 93%. All the other drugs (Atezolizumab, Nivolumab, Pembrolizumab, Pembrolizumab+Lenvatinib) showed 0% response (FIG. 7).

The perfusion system could have anticipated the unfavorable prognosis associated with Nivolumab (which was what was originally prescribed by the doctor) within 1-2 weeks, potentially sparing the patient six months of ineffective treatment, side effects, and the high costs as well as the time in which further mutations occurred. Additionally, the present evaluation revealed that the patient should be treated with Regorafenib, which is not typically indicated for the treatment of mucosal melanoma. Despite this discrepancy, and based on the results received by our 3D model, the patient was treated with Regorafenib and exhibited a notable clinical response, evidenced by a 50% reduction in tumor size within a two-week timeframe.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section 10headings are used, they should not be construed as necessarily limiting. In addition, any priority document(s) of this application is/are hereby incorporated herein by reference in its/their entirety.

What is claimed is:

1. A cell-perfusion system comprising:
a sequence of at least two cell-culture wells comprising:
a first cell-culture well which comprises a first population of cells;
a second cell-culture well which comprises a second, non-identical population of cells; and
a fluid pathway interconnecting said first cell-culture well and said second cell-culture well so as to allow for fluid to flow through the sequence in a sequential manner
wherein at least one of said first and said second population of cells comprises a plurality of cell types organized in a 3D cellular structure which models a tumor.

2. The system of claim 1, wherein said fluid pathway comprises at least one channel.

3. The system of claim 2, wherein said channel is lined with cellular matter.

4. The system of claim 3, wherein said cellular matter comprises cells selected from the group consisting of endothelial cells, pericytes and smooth muscles cells.

5. The system of claim 1, wherein at least a portion of said plurality of cell types are brain cells.

6. The system of claim 5, wherein said brain cells are selected from the group consisting of astrocytes, microglia cells, neuronal cells, and glioblastoma cells.

7. The system of claim 1, wherein at least a part of said cellular structure is generated by 3D printing.

8. The system of claim 1, wherein cells of said cellular structure are embedded in an extracellular matrix.

9. The system of claim 1, wherein said first population of cells comprises cancer cells and said second population of cells comprises lymph node cells.

10. The system of claim 1, wherein said first population of cells comprise cancer cells of a tissue of a first subject and said second population of cells comprise cancer cells of an identical tissue of a second subject.

11. The system of claim 1, wherein said first population of cells comprises cancer cells of a tumor and said second population of cells comprises metastasized cells of said tumor.

12. The system of claim 11, wherein said metastasized cells are selected from the group consisting of liver cells, lung cells, bone cells, and brain cells.

13. The system of claim 12, further comprising the fluid, wherein said fluid comprises blood cells.

14. The system of claim 13, wherein said first population or said second population of cells are derived from a subject and said fluid is derived from the subject.

15. The system of claim 1, wherein the sequence comprises at least three cell-culture wells, wherein a third culture well of said three cell-culture well comprises a third population of cells, wherein said second cell-culture well is connected to said third cell-culture well so as to allow for fluid to flow through the sequence in a sequential manner.

16. The system of claim 15, wherein said first population of cells comprise cancer cells of a tumor, said second population of cells comprise lymph node cells and said third population of cells comprise metastasized cells of said tumor.

17. A method of determining an effect of an agent comprising:
(a) flowing the agent through the cell perfusion system of claim 1; and
(b) measuring:
(i) at least one parameter of the fluid; and/or
(ii) at least one parameter of the cells of the first and/or second population;
wherein a change in said at least one parameter of the fluid and/or of the cells as compared to said at least one parameter in the absence of the agent, is indicative of an effect of the agent.

18. The method of claim 17, wherein said effect is a therapeutic effect.

19. The method of claim 17, wherein said effect is a prophylactic effect.

20. The method of claim 17, wherein said effect is a toxic effect.
